(19) United States
(12) Patent Application Publication
(10) Pub. No.: US 2012/0165957 A1
(43) Pub. Date: Jun. 28, 2012

(54) BIODEGRADABLE SCAFFOLD FOR SOFT TISSUE REGENERATION AND USE THEREOF

(76) Inventors: Hanne Everland, Bagsvaerd (DK); Lene Feldskov Nielsen, Copenhagen K (DK); Jakob Vange, Helsingoer (DK); Monica Ramos Gallego, Copenhagen SV (DK)

(21) Appl. No.: 13/382,359

(22) PCT Filed: Jul. 6, 2010
(86) PCT No.: PCT/DK10/50176

§ 371 Date: Mar. 8, 2012

(30) Foreign Application Priority Data
Jul. 6, 2009 (DK) PS2009 70054

(51) Int. Cl. A61F 2/02; A61F 13/02

(52) U.S. Cl. 623/23.72; 427/2.31

(57) ABSTRACT

The present invention relates to new reinforced biodegradable scaffolds for soft tissue regeneration, as well as methods for support and for augmentation and regeneration of living tissue, wherein a reinforced biodegradable scaffold is used for the treatment of indications, where increased strength and stability is required besides the need for regeneration of living tissue within a patient. The present invention further relates to the use of scaffolds together with cells or tissue explants for soft tissue regeneration, such as in the treatment of a medical prolapse, such as rectal or pelvic organ prolapse, or hernia.

### FIELD OF THE INVENTION

The present invention relates to new reinforced biodegradable scaffolds for soft tissue regeneration, as well as methods for support and for augmentation and regeneration of living tissue, wherein a reinforced biodegradable scaffold is used for the treatment of indications, where increased strength and stability is required besides the need for regeneration of living tissue within a patient. The present invention further relates to the use of scaffolds together with cells or tissue explants for soft tissue regeneration, such as in the treatment of a medical prolapse, such as rectal or pelvic organ prolapse, or hernia, or urinary incontinence.

### BACKGROUND OF THE INVENTION

Scaffolds are structures, such as synthetic polymer structures used to guide the organization, growth and differentiation of cells in the process of forming new functional tissue at the site of a tissue defect, wound, typically used in conjunction with surgical intervention.

To achieve the goal of tissue reconstruction, scaffolds must meet some specific requirements. A high porosity and an adequate pore size are necessary to facilitate cell growth and diffusion throughout the whole structure of both cells and nutrients. Biodegradability is essential since scaffolds need to be absorbed by the surrounding tissues without the necessity of a surgical removal.

Many different materials (natural and synthetic, biodegradable and permanent) have been investigated for use as scaffolds. Most of these materials have been known in the medical field before the advent of tissue engineering as a research topic, being already employed as bioresorbable sutures. Examples of these materials are collagen or some linear aliphatic polyesters.

Conditions like stress urinary incontinence and pelvic organ prolapse (POP) are indications for women seen as a result of multiparity, muscle weakness due to ageing and hormonal insufficiency. However, the same indications are also seen in younger inactive patients who have never given birth. Since the 1980's the use of synthetic meshes made from polypropylene has been in the preferred treatment. Examples of these meshes are: Prolene (Ethicon), Polyform (Boston Scientific) and Pelvitex (Bard). Over the last years, an increased number of side effects have been reported in up to 10% of the cases. Vaginal erosion and vaginal shortening are some of the more severe [“Rising use of synthetic mesh in transvaginal pelvic reconstructive surgery: A review of the risk of vaginal erosion”. E. Mistrangelo et. al., 3. Minimally Inasive Gynecology, 2007, 4, p. 564-69]

To overcome these side effects, a lighter version (less material) of the traditional mesh has been developed and some, which have been made partly degradable by combining polypropylene with a degradable synthetic polymer-like polylactide (Ultrapro, Ethicon). Cook Inc. has a xenografic approach which is completely degradable and based on decellurised extracellular matrix from porcine small intestines. [Mantovani F, Trinchieri A, Castelnuovo C, Romano A L, Pisani E. “Reconstructive urethroplasty using porcine acellular matrix.” Eur Urol 2003; 44:600-602.]

US2009024162 relates to absorbable composite medical devices such as surgical meshes and braided sutures, which display two or more absorption/biodegradation and breaking strength retention profiles.

WO08083394 relates to reinforced meshes for retropubic implants for treatment of urinary incontinence and/or pelvic floor disorders and related uses.

WO08042057 relates to devices for tissue reinforcement, more specifically devices having both a macroporous and microporous structure to allow both cell in-growth and tissue integration.

WO2006044881 and WO07117238 relates to a multilayered fabric comprising a first absorbable nonwoven fabric and a second absorbable woven or knitted fabric, and its method of manufacture.

EP 1674048 relates to a resorbable polymeric mesh implant that is intended to be used in the reconstruction of soft tissue defects. The mesh implant comprises at least a first and a second material, wherein the second material is substantially degraded at a later point in time than the first material following the time of implantation.

US 20080241213 relates to a biocompatible tissue implant, which may be bioabsorbable, and is made from biocompatible polymeric foam. The tissue implant also includes a biocompatible reinforcement member. The polymeric foam and the reinforcement member are soluble in a common solvent.

WO0222184 relates to tissue engineered prostheses made from processed tissue matrices derived from native tissues that are biocompatible with the patient or host in which they are implanted.

US2002062152 relates to medical bioresorbable implant particularly for crucial ligament augmentation constructed as a composite structure in textile construction from at least two biocompatible polymer materials which differ in their chemical composition and/or polymer structure and which are degradable the implant having a predetermined initial tensile stiffness and a different degradation behaviour of the polymers and/or the textile construction is selected in such a way that the tensile stiffness decreases during degradation.

WO06020922 relates to resorbable polylactide polymer scar tissue reduction barrier membranes and methods of their application are disclosed.

EP1216717 relates to bioabsorbable, porous, reinforced tissue engineered implant devices for use in the repair of soft tissue injury such as damage to the pelvic floor and methods for making such devices.

### OBJECT OF THE INVENTION

It is an object of embodiments of the invention to provide support to regenerate soft tissue by providing a fully degradable scaffold for in- or re-growth of either in vitro grown cells, cells/tissue harvested in the operating room or in- or re-growth of cells from the surrounding tissue.

Accordingly, scaffolds are provided with good properties with respect to tissue reconstruction, which at the same time are sufficiently strong to be suitable for implantation in medical conditions requiring structural support, such as injuries to tissue that require surgical intervention.

### SUMMARY OF THE INVENTION

It has been found by the present inventor(s) that a reinforced porous scaffold eases handling in the operation situation, ie. during surgery. Scaffolds made according to the invention provide sufficient strength during handling, combined with properties of stimulating regeneration of tissue of a patient requiring the implant and they are also strong enough to provide sufficient structural support at the site of regeneration.

It has to be understood that surgical implants being optimized for having properties for soft tissue regeneration in a patient are not always optimal for handling or for providing sufficient support at the site of the implant. This may be particularly relevant in medical conditions involving injuries to supporting structural soft tissue, such as in a medical prolapse, such as pelvic organ prolapse, stress urinary incontinence or hernias.

It has been found by the present inventors that structural support and reinforcement may be provided to the implant without compromising the ability to stimulate the regeneration of patient tissue at the site of injury.

So, in a first aspect, the present invention relates to a biodegradable surgical implant for support, augmentation and regeneration of living tissue in a subject, comprising
a) a synthetic biodegradable homogenous sheet of scaffold,
b) one or more biodegradable reinforcing members.

In a second aspect, the present invention relates to a method for support, augmentation and regeneration of living tissue within a subject, said method comprising implantation of a biodegradable surgical implant comprising a synthetic biodegradable scaffold together with a sample of autologous cells or tissue explants within said subject at the site wherein support, augmentation and regeneration of living tissue is required.

In a third aspect, the present invention relates to a method for the preparation of a biodegradable surgical implant comprising a synthetic biodegradable scaffold and autologous cells or tissue explants of a subject, suitable for support augmentation and regeneration of living tissue within said subject, said method comprising ex vivo application of a sample of said autologous cells or tissue explants on or within said biodegradable surgical implant comprising a synthetic biodegradable scaffold prior to implantation within said subject at the site wherein support, augmentation and regeneration of living tissue is required.

In a further aspect, the present invention relates to a biodegradable surgical implant comprising a synthetic biodegradable scaffold for use in a method for support, augmentation and regeneration of living tissue within a subject, said method comprising implantation of said biodegradable surgical implant comprising a synthetic biodegradable scaffold together with a sample of autologous cells or tissue explants within said subject at the site wherein support, augmentation and regeneration of living tissue is required.

In a further aspect, the present invention relates to a biodegradable surgical implant comprising a synthetic biodegradable scaffold, for use in a method for support, augmentation and regeneration of living tissue within a subject, said method comprising the steps of (i) extracting a tissue sample from the subject; (ii) disintegration or disruption of the tissue sample; (iii) implanting the scaffold and the crushed tissue sample into the subject.

In a further aspect, the present invention relates to a kit comprising
a) a biodegradable surgical implant comprising a synthetic biodegradable scaffold;
b) a sample of autologous cells or tissue explants; and
c) optionally, instructions for use in a method for support, augmentation and regeneration of living tissue within a subject, such as in a subject with a medical prolapse, such as rectal or pelvic organ prolapse, or hernia, said method comprising implantation of said biodegradable surgical implant together with an autologous sample of cells or tissue explants within said subject at the site wherein support, augmentation and/or regeneration of living tissue is required.

In a further aspect the present invention relates to a kit comprising
a) a synthetic biodegradable scaffold; and
b) a device suitable for disintegration or disruption of a tissue sample.

In a further aspect, the present invention relates to a method for support, augmentation and regeneration of living tissue within a subject with a medical prolapse, such as pelvic organ prolapse, or hernia, the method comprising implantation of a biodegradable surgical implant comprising a synthetic biodegradable homogenous sheet of scaffold together with a sample of cells or tissue explants within the subject at the site of the prolapse or hernia.

In a further aspect, the present invention relates to a method for support, augmentation and regeneration of living tissue within a subject, the method comprising implantation of biodegradable surgical implant for support, augmentation and regeneration of living tissue in a subject, comprising
a) a synthetic biodegradable homogenous sheet of scaffold,
b) one or more biodegradable reinforcing members;

characterised in that the synthetic biodegradable homogenous sheet of scaffold is hydrophilic within the subject.

In a further aspect, the present invention relates to a method for the preparation of a biodegradable surgical implant according to the invention, which method comprises the simultaneous of sequential steps of
a) preparing the synthetic biodegradable homogenous sheet of scaffold;
b) preparing and incorporating the one or more biodegradable reinforcing members within the synthetic biodegradable homogenous sheet of scaffold;
c) optionally incorporating one or more components as defined herein.

In a further aspect, the present invention relates to a kit comprising
a) biodegradable surgical implant according to the invention;
b) a sample of cells or tissue explants; and
c) optionally, instructions for use in a method for support, augmentation and regeneration of living tissue within a subject with a medical prolapse, such as pelvic organ prolapse, or hernia, the method comprising implantation of the biodegradable surgical implant together with a sample of cells or tissue explants within the subject at the site of the prolapse or hernia.

In a further aspect, the present invention relates to an implant according to the invention for use as a medicament.

In a further aspect, the present invention relates to an implant according to the invention for use in the treatment of a disease related to pelvic organ prolapse and hernia.

### LEGENDS TO THE FIGURES

FIG. 1: SEM picture of a cross section of a scaffold made by freeze-drying. The orientation of the material is along the direction of freezing.

FIG. 2a: 40×40 mm scaffolds. Left: unmodified. Right: welded in edges for added strength.

FIG. 2b: scaffold welded in grid pattern for added strength

FIG. 3: Scaffold welded to backing of electrospun PLGA.

FIG. 4: Freeze-dried structure reinforced by inclusion of a grid of suture.

FIG. 5: Illustration depicts different patterns that can be used in order to reinforce the scaffold by the inclusion of biodegradable threads.

FIG. 6: Regarding the flexibility of the scaffold. It is depicted in this figure that when the scaffold made of mPEG-PLGA is dry, it is rigid. On the other hand, once it is wet it becomes very pliable. This compared to the polypropylene mesh, which does not become less rigid, after exposure to water.

FIG. 7: 200, 300 and 400 μm ss mesh soldered to 8 mm ss washers.

FIG. 8: Treaded high pressure device (thread not shown)

FIG. 9: The flap; Biodegradable surgical implant comprising a scaffold.

FIG. 10: Left: cells are applied, Right: flap is closed and optionally sutured.

FIG. 11: Full-length or segmented flaps.

FIG. 12: The tube; Biodegradable surgical implant comprising a scaffold designed as a tube.

FIG. 13: The pocket; Biodegradable surgical implant comprising a scaffold designed with a pocket.

FIG. 14: Absorbent 3D scaffold welded to a backing material.

FIG. 15: Wetting of E-spun sheets with blood (15 minutes). Left: PCL coaxially coated with MPEG-PLGA 2-30 50DL. Right: plain PCL.

FIG. 16: A: Porous sponge structure of MPEG-PLGA. Dashed line marks the edge between the surface and the cross-sectional view of the implant. B: The knitted structure of the Vicryl mesh. Digital images of dark-field stereomicroscopy at 10× magnification. Scale bar: 1.0 mm.

FIG. 17: MPEG-PLGA combined with fragmented muscle fibres after 3 weeks. Muscle tissue located beneath the implant.

FIG. 18: MPEG-PLGA combined with fragmented muscle fibres after 8 weeks. Muscle tissue located where implant and fragmented muscle fibres were previously implanted.

FIG. 19: biodegradable surgical implant with arms/extensions for attachment to structures in the pelvic region.

### DETAILED DISCLOSURE OF THE INVENTION

In the present context, the terms “biodegradable”, “bioabsorbable” and just “degradable” as used herein refers to a polymer that disappears over a period of time after being introduced into a biological system, which may be in vivo (such as within the human body) as in the present invention, or in vitro (when cultured with cells); the mechanism by which it disappears may vary, it may be hydrolysed, be broken down, be biodegraded, be bioresorbed, be bioabsorbed, be bioerodable, be dissolved or in other ways vanish from the biological system. When used within a clinical context this is a huge clinical advantage as there is nothing to remove from the site of repair. Thus, the newly formed tissue is not disturbed or stressed by presence of or even the removal of the temporary scaffold. In some embodiments, the scaffold is broken down during 1 day to 4 years, such as 1 day to a year, such as during 2 to 6 months.

The term “biocompatible” refers to a composition or compound, which, when inserted into the body of a mammal, such as the body of a patient, particularly when inserted at the site of the defect, does not lead to significant toxicity or a detrimental immune response from the individual.

When the term “about” is used herein in conjunction with a specific value or range of values, the term is used to refer to both the range of values, as well as the actual specific values mentioned.

The term “culturing in vitro”, as used herein, refers to the step of the method according to the invention, wherein a sample of cells or tissue explants are maintained under in vitro conditions, i.e. under conditions of a controlled environment outside of a living mammal. Alternatively, the skilled person may use the phrases that the “cells are grown”, or “cells are proliferated” in vitro, which is also within the meaning of “culturing”.

The term “elongation at break” as used herein refers to % elongation, wherein the scaffold polymer or reinforced surgical implant according to the invention will break as measured by the assay described in example 3.

The term “tensile strength” as used herein refers to strength of the scaffold polymer or reinforced surgical implant according to the invention as measured in N/m² or psi by the assay described in example 3.

The phrase “vertical pore structure” as used herein refers to the pore structure of the scaffold polymer used according to the invention, wherein the pores primarily are oriented in a vertical direction to the sheet of scaffold. This will allow for a better absorption of liquids and cells at the site of implantation.

The term “interconnected pores” as used herein refers to scaffold polymer used according to the invention that has a pore structure with openings between individual pores, such as openings in a horizontal direction between individual pores with a primarily vertical orientation. This will allow cells to migrate in any direction through the scaffold polymer material.

The term “tissue” as used herein refers to a solid living tissue which is part of a living mammalian individual, such as a human being. The tissue may be a hard tissue (e.g. bone, joints and cartilage) or soft tissue including tendons, ligaments, fascia, fibrous tissues, fat, and synovial membranes, and muscles, nerves and blood vessels.

In particular aspects, a sample of cells or tissue explants, such as a body fluid sample optionally mixed with culture medium, are placed on the surface of or at least in conjunction with the scaffold, usually in a culture dish or flask. The sample of cells or tissue explants may be placed together with a component which facilitates the cell adhesion, re-growth, and/or in-growth through the scaffold.

In another aspect, muscle biopsies are placed in a container with an appropriate buffer e.g. cell media, PBS, etc. Cells and muscle fibres are isolated from the biopsies by the use of a tissue grinder (e.g. Sigma-Aldrich). Afterwards, the muscle suspension is applied to the surface of the scaffold before or concomitantly with the implantation.

The muscle suspension used according to aspects of the invention is typically seeded with a density in the range of 1-100 mg muscle suspension per cm² of scaffold sheet.

In another aspect, muscle fibres are isolated from biopsies either by dissecting the muscle with e.g. scalpels or dissolution of the muscle using enzymatic treatment e.g. collagenase, to get single fibres with satellite cells. These fibres are applied to the surface of the scaffold before implantation.

Accordingly, tissue explants from muscle tissue may be from muscle dissected into a muscle puree by e.g. scalpels or wherein muscle fibres are isolated from the remaining tissue using mechanically or enzymatic methods, or wherein the muscle tissue is ground into a muscle slurry, all of which comprises a population of myoblasts and fibroblasts, and/or muscle precursor cells like satellite cells.

It is to be understood that once the body fluid sample has been applied to the synthetic biodegradable scaffold, cells in situ at the place of medical application, or alternatively cells contained within the body fluid sample are allowed to migrate and/or grow through the scaffold to generate new tissue, such as new connective and/or muscle tissue. In one embodiment, a component which facilitates cell adhesion and/or in-growth is concomitantly applied to the scaffold.

The Scaffold

The synthetic biodegradable scaffold used according to the present invention is a porous structure that stimulates and facilitates growth of tissue and cells. The scaffold is made up of biocompatible, degradable materials and are used in the implant to guide the organization, growth and differentiation of cells in the process of forming functional tissue at the site of an injury in a patient.

In most aspects of the invention, the synthetic biodegradable scaffold is completely or partially degraded in situ at the place of a medical application within a period of up to about 48 months, such as within a period of up to about 36 months, such as within a period of up to about 24 months, such as within a period of up to about 12 months, such as within a period of up to about 10 months, such as within a period of up to about 9 months, such as within a period of up to about 6 months, such as within a period of up to about 5 months, such as within a period of up to about 4 months, such as within a period of up to about 3 months, such as within a period of up to about 2 months, such as within a period of up to about 1 month as measured after the medical application.

In some important aspects of the invention, the synthetic biodegradable scaffold is not completely or partially degraded in situ at the place of surgical application until after a period of about 1 month, such as after a period of about 2 months, such as after a period of about 3 months, such as after a period of about 4 months, such as after a period of about 5 months, such as within a period of about 6 months, such as after a period of about 9 months such as within a period of about 12 months, such as within a period of about 24 months, such as within a period of about 36 months, as measured after the medical application.

The phrase “completely or partially degraded in situ” refers to the synthetic biodegradable scaffold being degraded at the place of the medical application by the action of intrinsic components of the body or extrinsic components of the scaffold or body fluid sample applied to the scaffold. This action may be endogenous enzymatic activity of the body fluids or alternatively by the activity of compounds added to the scaffold.

In some embodiments, the synthetic biodegradable scaffold is degraded to a level of at least about 50%, such as at least about 60%, such as at least about 70%, such as at least about 70%, such as at least about 80%, such as at least about 90%, such as at least about 100% within a given period of time.

It is to be understood that the scaffold material, with an inherent rate of degradation may be selected to fit the time necessary for providing sufficient support and reinforcement at the site of a medical application until the patient's own tissues provide necessary support and strength.

In some embodiments the synthetic biodegradable scaffold is selected to be completely or partially degradable by cellular degradation, i.e. degraded by the action of cell enzymes, such as enzyme action of the patient's body fluids.

It is to be understood that the scaffold material sensitive to cellular degradation may be selected to fit a specific and suitable period of degradation.

In some embodiments the synthetic biodegradable scaffold is sterilised through the application of irradiation, such as beta radiation, or plasma sterilisation.

The synthetic biodegradable scaffold, before being implanted may be cut or “sized” to fit a particular defect—suitably the scaffold may be cut to a particular shape or form to suit the site of a particular defect and/or the desired shape/form of a new tissue.

The synthetic biodegradable scaffold may be used in one or several layers, as fibres, woven and/or non-woven materials, such as with a porous structure.

In some embodiments the scaffold is biocompatible.

In one embodiment, the scaffold comprises a polymer, which may be selected from the group consisting of: collagen, alginate, polylactic acid (PLA), polyglycolic acid (PGA), MPEG-PLGA or PLGA.

In one embodiment, the scaffold comprises a polymer, which may be selected from the group consisting of: 1) Homo- or copolymers of: glycolide, L-lactide, DL-lactide, meso-lactide, e-caprolactone, 1,4-dioxane-2-one, d-valerolactone, β-butyrolactone, g-butyrolactone, e-decalactone, 1,4-dioxepane-2-one, 1,5-dioxepane-2-one, 1,5,8,12-tetraoxacyclotetradecane-7-14-dione, 6,6-dimethyl-1,4-dioxane-2-one, and trimethylene carbonate; 2) Block-copolymers of mono- or difunctional polyethylene glycol and polymers of 1) mentioned above; 3) Block copolymers of mono- or difunctional polyalkylene glycol and polymers of 1) mentioned above; 4) Blends of the above mentioned polymers; and 5) polyanhydrides and polyorthoesters.

In some embodiments the scaffold has the ability of being hydrophilic. Accordingly, the scaffold is wettable to water, isotonic buffers and/or blood and other body fluids.

In one embodiment, the scaffold essentially consists of or comprises a polymer, or polymers, of molecular weight, such as average molecule weight, greater than about 1 kDa, such as between about 1 kDa and about 1 million kDa, such as between 25 kDa and 100 kDa.

The scaffold is preferably made as a sheet, which is suitable for implantation in the diaphragm, abdomen, or the pelvic floor region.

The scaffold sheet may be selected from the group consisting of: a membrane, such as a porous membrane, a sheet, such as a porous sheet, sheet of fibres, the sheet may have various 2 dimensional forms, such as a custom made implant for insertion onto the site of defect, such as to fit in a surgical reconstruction of fascia of the mammalian body, a foam, the sheet may be woven or non-woven, freeze-dried polymer such as freeze-dried polymer sheets or any combination of these.

Alternatively, the scaffold may be a custom made three dimensional construct of desired shape fitted for implantation into the site of defect or site requiring implantation.

Suitably, scaffolds may be of any type and size, as well as any thickness of a scaffold, such as ranging from thin membranes to several millimetre thick scaffolds, such as in the range of about 0.1 mm to 6 mm, such as in the range of about 0.2 mm to 6 mm, such as in the range of about 0.5 mm to 6 mm.

In one embodiment, the scaffold is in the form of a sheet, which may be pre-cut or sized to fit the defect. Such a scaffold may be, for example between about 0.2 mm to 6 mm thick.

The pores of the scaffold may be partly occupied by a component which facilitates the cell adhesion and/or in-growth for regeneration of tissue, such as a component selected from the group consisting of: estrogen, estrogen derivatives, ECM powder, thrombin, chondroitin sulfate, hyaluronan, heparin sulfate, heparan sulfate, dermatan sulfate, growth factors, fibrin, fibronectin, elastin, collagen, gelatin, and aggrecan. Alternatively, the components may be totally or partially incorporated or embedded within the scaffold.

As discussed above, the scaffolds may consist or comprise any suitable biologically acceptable material, however in a preferred embodiment the scaffold comprises a compound selected from the group consisting of: polylactide (PLA), polycaprolacttone (PCL), polyglycolide (PGA), poly(D,L-lactide-co-glycolide) (PLGA), MPEG-PLGA (methoxypolyethyleneglycol)-poly(D,L-lactide-co-glycolide), polyhydroxyacids in general. In this respect the scaffold, excluding the pore space and any additional components, such as those which facilitate the cell adhesion and/or in-growth for regeneration of tissue, may comprise at least 50%, such as at least 60%, at least 70%, at least 80% or at least 90%, of one or more of the polymers provided herein, including mixtures of polymers.

In some embodiments, the scaffold and reinforcing member is made of polycaprolactone (PCL), such as electrospun PCL, copolymers of caprolactone and lactide or biodegradable polyurethanes

PLGA and MPEG-PLGA are suitable scaffold materials.

In one embodiment, the synthetic biodegradable scaffold is a scaffold as prepared by the method disclosed in WO 07/101,443. The method is particularly suited to prepare scaffolds from PLGA and MPEG-PLGA polymers.

In some aspects of the present invention, the synthetic biodegradable scaffold is a scaffold prepared by the method disclosed in WO 07/101,443, which method comprises the steps of:
(a) dissolving a polymer as defined herein in a non-aqueous solvent so as to obtain a polymer solution;
(b) freezing the solution obtained in step (a) so as to obtain a frozen polymer solution; and
(c) freeze-drying the frozen polymer solution obtained in step (b) so as to obtain the biodegradable, porous material.

The non-aqueous solvent used in the method as disclosed in WO 07/101,443 should, with respect to melting point be selected so that it can be suitably frozen. Illustrative examples hereof are dioxane (mp. 12° C.) and dimethylcarbonate (mp. 4° C.).

In one variant of the method as disclosed in WO 07/101,443, the polymer solution, after step (a) above, is poured into or cast in a suitable mould. In this way, it is possible to obtain a three-dimensional shape of the material specifically designed for the particular application.

In embodiments, wherein particles of components from the extracellular matrix are used in the methods according to the invention, these extracellular matrix components may be dispersed in the solution obtained in step (a) before the solution (dispersion) is frozen as defined in step (b).

The components from the extracellular matrix may, for instance, be dissolved in a suitable solvent and then added to the solution obtained in step (a). By mixing with the solvent of step (a), i.e. a solvent for the polymer defined herein, the components from the extracellular matrix will most likely precipitate so as to form a dispersion.

In one aspect, the biodegradable, porous material obtained in step (c), in a subsequent step, is immersed in a solution of glucosaminoglycan (e.g. hyaluronan) and subsequently freeze-dried again.

In some alternative embodiments, the materials are present in the form of a fibre or a fibrous structure prepared from the polymer defined herein, possibly in combination with components from the extracellular matrix. Fibres or fibrous materials may be prepared by techniques known to the person skilled in the art, e.g. by melt spinning, electrospinning, extrusion, etc.

In preferred embodiments, the synthetic biodegradable scaffold is biocompatible. Even if the scaffold structure according to the invention is degraded, scaffold degradation products may still be present at the site of original implant. Accordingly, it may still be an advantage to use biocompatible scaffold material.

The porous scaffold material may be prepared according to known techniques, e.g. as disclosed in Antonios G. Mikos, Amy J. Thorsen, Lisa A Cherwonka, Yuan Bao & Robert Langer. Preparation and characterization of poly(L-lactide) foams. Polymer 35, 1068-1077 (1994). One very useful technique for the preparation of the porous materials is, however, freeze-drying.

In some embodiments, the scaffold has porosity in the range of 20% to 99%, such as at least 50%, such as 50 to 95%, or 75% to 95% or to 99%.

The high degree of porosity can be obtained by freeze-drying.

In some embodiments, the surgical implant according to the invention does not comprise a biological polymer, i.e. a biopolymer such as protein, polysaccharide, polyisoprenes, lignin, polyphosphate or polyhydroxyalkanoates.

In other embodiments, the scaffold further comprises a biological polymer, i.e. a biopolymer, such as polypeptide, protein, polysaccharide, lignin, polyphosphate or polyhydroxyalkanoates (e.g. as described in U.S. Pat. No. 6,495,152). Suitable biopolymers may be selected from the group consisting of: gelatin, hyaluronan, hyaluronic acid (HA), chondroitin sulphate, dermatan sulphate, collagen, such as collagen type I and/or type II, alginate, alginate, chitin, chitosan, keratin, silk, elastin, cellulose and derivatives thereof.

The scaffold may be prepared by freeze-drying a solution comprising the compound, such as those listed above, in solution.

The components from the extracellular matrix could be added either as particles, which are heterogeneously dispersed, or as a surface coating. The concentration of the components from the extracellular matrix relative to the synthetic polymer is typically in the range of 0.5-15% (w/w), such as below 10% (w/w). Moreover, the concentration of the components of the extracellular matrix is preferably at the most 0.3% (w/v), e.g. at the most 0.2 (w/v), relative to the volume of the material.

The required type of scaffolds used within the context of this invention shall be scaffolds that do not act as foreign bodies in the mammal (including humans) so that no immunity or a minimum of immunity may be observed and the scaffolds used in this context shall not be toxic or significantly harmful to the organism in which they are placed. Preferably, the scaffold does not contain any microbial organisms, or any other harmful contaminants.

Cells or tissue explants used in the scaffold may be embedded in a hydrogel, and may be capable of being placed onto the scaffold, before the scaffold is placed in its target area. The scaffold may be hydrophilic so that the cell material is absorbed relatively quickly into the scaffold. In other suitable embodiments, cell material is placed within a pocket, under a flap, or within a tube of scaffold material.

The scaffold may be hydrophilic, i.e. have the ability to, within 5 minutes, such as within 2 minutes at 30° C. to absorb at least a small amount of water or aqueous solution (such as the cell suspension composition), such as absorb at least 1%, such as at least 2%, such as at least 5%, such as at least 10%, such as at least 20%, such as at least 30%, such as at least 50% of the scaffold volume of water (or equivalent aqueous solution) when placed in an aqueous solution, such as a physiological media, a buffer, water, blood or other body fluid, it is particularly beneficial that the scaffold can absorb the above amounts of the cell suspension into its porous structure, thereby providing a relatively homogenous distribution of cells, such as endogenous cells or in vitro applied cells or tissue explants throughout the scaffold once inserted and fixed into the site of defect.

In other embodiments, a scaffold material is determined as “hydrophilic” by a method, wherein a drop of plasma or blood is placed on top of the sheet of scaffold material; the bottom of the sheet of scaffold material is observed; and the sheet of scaffold material is considered hydrophilic if breakthrough of liquid is seen within 15 minutes.

In some embodiments, the biodegradable polymer is at least partly hydrophilic, i.e. has a component of the polymer, which may reasonably be considered hydrophilic, such as an MPEG part of an MPEG-PLGA co-polymer.

The term “hydrophilic” is used interchangeably with the term ‘polar’.

One way to improve hydrophilicity of the scaffold polymer is a pre-treatment with an agent which facilitates the uptake of endogenous cells at the site of the implant or cells applied to the scaffold prior to implantation, such as anionic, cationic, non-ionic detergents or amphilic detergents, buffers or salts. Wetting agents may also be used in conjunction with hydrophilic scaffolds to further improve cell penetration into the porous structure.

The biocompatible scaffold of the invention may comprise polyesters. By incorporating a balanced hydrophilic block in the polymer, the biocompatibility of the polymer may be improved as it improves the wetting characteristics of the material, and initial cell adhesion is impaired on non-polar materials.

In one important aspect of the invention, the scaffold is biodegradable.

In some embodiments the scaffold is porous, e.g. has a porosity of at least 25%, 50%, such as in the range of 50-99.5%. Porosity may be measured by any method known in the art, such as comparing the volume of pores compared to the volume of solid scaffold. This may be done by determining the density of the scaffold as compared to a non-porous sample having the same composition as the scaffold. Alternatively Mercury Intrusion Porosimetry or BET may be used.

In a highly interesting embodiment of the invention, the biocompatible scaffold according to the invention consists of or comprises of one or more of the polymers selected from the group comprising: poly(L-lactic acid) (PLLA), poly(D/L-lactic acid) (PDLLA), Poly(caprolactone) (PCL) and poly(lactic-co-glycolic acid) (PLGA), and derivatives thereof, particularly derivatives which comprise the respective polymer backbone, with the addition of substituent groups or compositions which enhance the hydrophilic nature of the polymer e.g. MPEG or PEG. Examples are provided herein, and include a group of polymers, MPEG-PLGA

In one embodiment, the scaffold consists of or comprises a synthetic polymer.

Polymers Used in the Preparation of the Scaffold

WO 07/101,443 discloses suitable polymers for use as scaffold materials in the present invention as well as methods for their preparation.

Suitable biodegradable polymers for use in the method of the invention are composed of a polyalkylene glycol residue and one or two poly(lactic-co-glycolic acid) residue(s).

Hence, in one aspect of the present invention the scaffold is prepared from, or comprises or consists of a polymer of the general formula:

A-O—(CHR¹CHR²O)ₙ—B

wherein

A is a poly(lactide-co-glycolide) residue of a molecular weight of at least 4000 g/mol, the molar ratio of (i) lactide units and (ii) glycolide units in the poly(lactide-co-glycolide) residue being in the range of 80:20 to 10:90, in particular 70:30 to 10:90, 60:40 to 40:60, such as about 50:50, such as 50:50;

B is either a poly(lactide-co-glycolide) residue as defined for A or is selected from the group consisting of hydrogen, C₁₋₆-alkyl and hydroxy protecting groups,

one of R¹ and R² within each —(CHR¹CHR²O)— unit is selected from hydrogen and methyl, and the other of R¹ and R² within the same —(CHR¹CHR²O)— unit is hydrogen,

n represents the average number of —(CHR¹CHR²O)— units within a polymer chain and is an integer in the range of 10-1000, in particular 16-250,

the molar ratio of (iii) polyalkylene glycol units —(CHR¹CHR²O)— to the combined amount of (i) lactide units and (ii) glycolide units in the poly(lactide-co-glycolide) residue(s) is at the most 20:80, and wherein the molecular weight of the copolymer is at least 10,000 g/mol, preferably at least 15,000 g/mol, or even at least 20,000 g/mol.

Hence, the polymers for use in the method of the invention can either be of the diblock-type or of the triblock-type.

In some important aspects of the invention, the synthetic biodegradable scaffold is designed to have a specific rate of degradation in vitro. This may be accomplished by varying the individual components (or ratios individual components) within the polymer.

In some embodiments, the degradation time is varied by the G-L-ratio and molecular weight of MPEG-PLGA polymers: It is possible to vary the degradation time of copolymers of DL-lactide and glycolide by varying the molar ratio of lactide and glycolide. Pure polyglycolide has a degradation time of 6-12 months, poly(D,L-lactide): 12-16 months, poly(D,L-lactide-co-glycolide 85:15 molar ratio: 2-4 months. The shortest degradation is obtained with a 50:50 molar ratio: 1-2 months. It is also possible to vary the degradation time by varying the molecular weight, but this effect is small compared to the variations possible with the L:G-ratio. In theory it is possible to get substantially faster degradation with very low molecular weight materials, but these have mechanical properties that preclude their use for most medical devices.

In one particular embodiment A in the above formula is a poly(lactide-co-glycolide) residue of a molecular weight of at least 4000 g/mol, the molar ratio of (i) lactide units and (ii) glycolide units in the poly(lactide-co-glycolide) residue being in the range of approximately 50:50 molar ratio.

The porosity of the polymer may be at least 50%, such as in the range of 50-99.5%.

It is understood that the polymer for use in the method of the invention comprises either one or two residues A, i.e. poly(lactide-co-glycolide) residue(s). It is found that such residues should have a molecular weight of at least 4000 g/mol, more particularly at least 5000 g/mol, or even at least 8000 g/mol.

The poly(lactide-co-glycolide) of the polymer can be degraded under physiological conditions, e.g. in body fluids and in tissue. However, due to the molecular weight of these residues (and the other requirements set forth herein), it is believed that the degradation will be sufficiently slow so that materials and objects made from the polymer can fulfil their purpose before the polymer is fully degraded.

The expression “poly(lactide-co-glycolide)” encompasses a number of polymer variants, e.g. poly(random-lactide-co-glycolide), poly(DL-lactide-co-glycolide), poly(mesolactide-co-glycolide), poly(L-lactide-co-glycolide), poly(D-lactide-co-glycolide), the sequence of lactide/glycolide in the PLGA can be either random, tapered or as blocks and the lactide can be either L-lactide, DL-lactide or D-lactide.

Preferably, the poly(lactide-co-glycolide) is a poly(random-lactide-co-glycolide) or poly(tapered-lactide-co-glycolide).

Another important feature is the fact that the molar ratio of (i) lactide units and (ii) glycolide units in the poly(lactide-co-glycolide) residue(s) should be in the range of 80:20 to 10:90, in particular 70:30 to 10:90.

It has generally been observed that the best results are obtained for polymers wherein the molar ratio of (i) lactide units and (ii) glycolide units in the poly(lactide-co-glycolide) residue(s) is 70:20 or less. However, fairly good results were also observed when, for polymer having a respective molar ratio of up to 80:20 as long as the molar ratio of (iii) polyalkylene glycol units —(CHR¹CHR²O)— to the combined amount of (i) lactide units and (ii) glycolide units in the poly(lactide-co-glycolide) residue(s), was at the most 8:92.

As mentioned above, B is either a poly(lactide-co-glycolide) residue as defined for A or is selected from the group consisting of hydrogen, C₁₋₆-alkyl and hydroxy protecting groups.

In one embodiment, B is a poly(lactide-co-glycolide) residue as defined for A, i.e. the polymer is of the triblock-type.

In another embodiment, B is selected from the group consisting of hydrogen, C₁₋₆-alkyl and hydroxy protecting groups, i.e. the polymer is of the diblock-type.

Most typically (within this embodiment), B is C₁₋₆-alkyl, e.g. methyl, ethyl, 1-propyl, 2-propyl, 1-butyl, tert-butyl, 1-pentyl, etc., most preferably methyl. In the event where B is hydrogen, i.e. corresponding to a terminal OH group, the polymer is typically prepared using a hydroxy protecting group as B. “Hydroxy protecting groups” are groups that can be removed after the synthesis of the polymer by e.g. hydrogenolysis, hydrolysis or other suitable means without destroying the polymer, thus leaving a free hydroxyl group on the PEG-part, see, e.g. textbooks describing state-of-the-art procedures such as those described by Greene, T. W. and Wuts, P. G. M. (Protecting Groups in Organic Synthesis, third or later editions). Particularly useful examples hereof are benzyl, tetrahydropyranyl, methoxymethyl, and benzyloxycarbonyl. Such hydroxy protecting groups may be removed in order to obtain a polymer wherein B is hydrogen.

One of R¹ and R² within each —(CHR¹CHR²O)— unit is selected from hydrogen and methyl, and the other of R¹ and R² within the same —(CHR¹CHR²O)— unit is hydrogen. Hence, the —(CHR¹CHR²O)ₙ— residue may either be a polyethylene glycol, a polypropylene glycol, or a poly(ethylene glycol-co-propylene glycol). Preferably, the —(CHR¹CHR²O)ₙ— residue is a polyethylene glycol, i.e. both of R¹ and R² within each unit are hydrogen.

n represents the average number of —(CHR¹CHR²O)— units within a polymer chain and is an integer in the range of 10-1000, in particular 16-250. It should be understood that n represents the average of —(CHR¹CHR²O)— units within a pool of polymer molecules. This will be obvious for the person skilled in the art. The molecular weight of the polyalkylene glycol residue (—(CHR¹CHR²O)ₙ—) is typically in the range of 750-10,000 g/mol, e.g. 750-5,000 g/mol.

The —(CHR¹CHR²O)ₙ— residue is typically not degraded under physiological conditions, but may, on the other hand, be secreted in vivo, e.g. from the human body.

The molar ratio of (iii) polyalkylene glycol units —(CHR¹CHR²O)— to the combined amount of (i) lactide units and (ii) glycolide units in the poly(lactide-co-glycolide) residue(s) also plays a certain role and should be at the most 20:80. More typically, the ratio is at the most 18:82, such as at the most 16:84, preferably at the most 14:86, or at the most 12:88, in particular at the most 10:90, or even at the most 8:92. Often, the ratio is in the range of 0.5:99.5 to 18:82, such as in the range of 1:99 to 16:84, preferably in the range of 1:99 to 14:86, or in the range of 1:99 to 12:88, in particular in the range of 2:98 to 10:90, or even in the range of 2:98 to 8:92.

It is believed that the molecular weight of the copolymer is not particularly relevant as long as it is at least 10,000 g/mol. Preferably, however, the molecular weight is at least 15,000 g/mol. The “molecular weight” is to be construed as the number average molecular weight of the polymer, because the skilled person will appreciate that the molecular weight of polymer molecules within a pool of polymer molecules will be represented by values distributed around the average value, e.g. represented by a Gaussian distribution. More typically, the molecular weight is in the range of 10,000-1,000,000 g/mol, such as 15,000-250,000 g/mol. or 20,000-200,000 g/mol. Particularly interesting polymers are found to be those having a molecular weight of at least 20,000 g/mol, such as at least 30,000 g/mol.

The polymer structure may be illustrated as follows (where R is selected from hydrogen, C₁₋₆-alkyl and hydroxy protecting groups; n is as defined above, and m, p and ran are selected so that the above-mentioned provisions for the poly(lactide-co-glycolide) residue(s) are fulfilled):

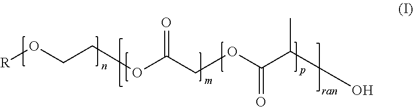

diblock-type polymer

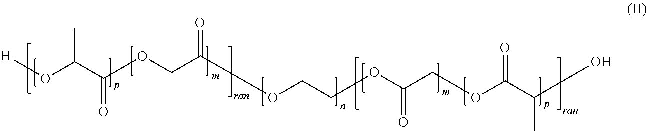

triblock-type polymer

For each of the above-mentioned polymer structures (I) and (II), it will be appreciated that the lactide and glycolide units represented by p and m may be randomly distributed depending on the starting materials and the reaction conditions.

Also, it is appreciated that the lactide units may be either D/L or L or D, typically D/L or L.

As mentioned above, the poly(lactide-co-glycolide) residue(s), i.e. the polyester residue(s), is/are degraded hydrolytically in physiological environments, and the polyalkylene glycol residue is secreted from, e.g. the mammalian body. The biodegradability can be assessed as outlined in the Experimentals section.

The polymers can in principle be prepared following principles known to the person skilled in the art.

In principle, polymer where B is not a residue A (diblock-type polymers) can be prepared as follows:

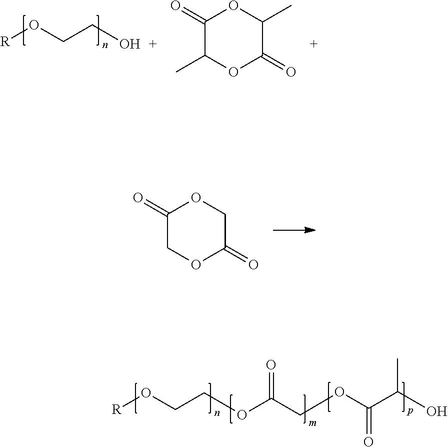

In principle, polymer where B is a residue A (triblock-type polymers) can be prepared as follows:

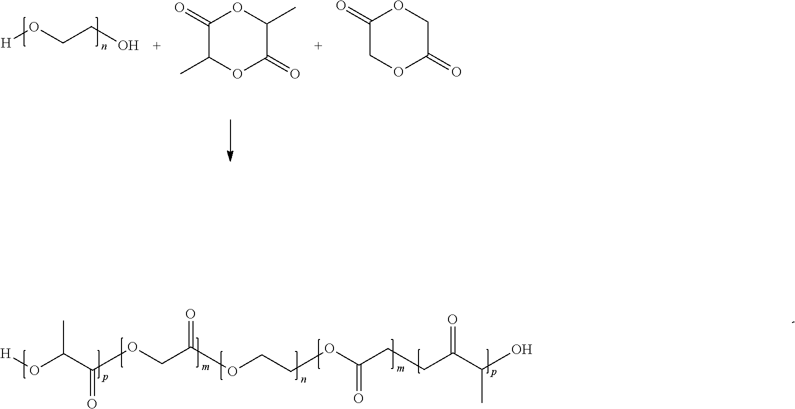

Unless special conditions are applied, the distribution of lactide units and glycolide units will be randomly distributed or tapered within each poly(lactide-co-glycolide) residue.

Preferably the ratio of glycolide units and lactide units present in the polymer used in scaffold is between an upper limit of about 80:20, and a lower limit of about 10:90, and a more preferable range of about 60:40 to 40:60.

Preferably the upper limit of PEG-content is at most about 20 molar %, such as at most about 15 molar %, such as between 1-15 molar %, preferably between 4-9 molar %, such as about 6 molar %.

The synthesis of the polymers according to the invention is further illustrated in the international patent application WO 07/101,443, the content of which is hereby incorporated by reference in its entirety.

In some embodiments, the scaffold polymer used according to the invention has a vertical pore structure. In some embodiments, the vertical open pore structure is with a significant number of openings in a horizontal direction between individual pores, i.e. interconnected pores.

Reinforcing Member of the Implant

As discussed elsewhere, the biodegradable scaffold used in the implant of the present invention is reinforced to serve the purpose of providing the implant with the required reinforcement for easily applying the implant. Accordingly, a reinforcing member may have a higher tensile strength than a scaffold used in the implant of the present invention.

The reinforcing member may be in the form of a second polymer, which is different from the polymer of the scaffold. In this aspect, the reinforcing member may have a degradation time, which is different from the degradation time of the scaffold.

Alternatively and in particular aspects, the reinforcing member is made of the same polymer as the scaffold. In these aspects, the strength is provided by having the polymer as fibres or as a fibrous material prepared by techniques known to the person skilled in the art, e.g. by melt spinning, electrospinning, extrusion, etc. Alternatively, the strength is provided by welding seams of the scaffold polymer material. The reinforcing member may have a degradation time, which is different from the degradation time of the scaffold, however it may also be similar or close to the same.

The density and volume of the reinforcing member should be sufficient to provide the needed reinforcement for easily handling the implant and functionality. In some embodiments the volume % should be sufficient to permit suturing within the implant and without destroying the implant. However, the volume % should not be as great as to impede flexibility or to compromise the ability of the implant to support regeneration of the tissue. Accordingly, in some embodiments the volume % of the reinforcing member is in the range of less than about 12%, such as less than about 10%, such as less than about 8%.

The “volume %” of the reinforcing member may be evaluated using image analysis. The volume is given as the percentage of the total volume taken up by the reinforcing member(s).

In some embodiments, the reinforced implant according to the present invention is flexible when wetted to saturation with a liquid as measured by the flexibility assay as described in example 2.

Accordingly, the term “flexible” as used herein refers to the ability of the implant or scaffold in the size of 1-2 cm² to bend when taken with tweezers.

Suitable polymers to be used as reinforcing member is a polymer made from a polymer of poly(lactide-co-glycolide) PLGA, such as a polymer wherein the molar ratio of (i) lactide units and (ii) glycolide units in the poly(lactide-co-glycolide) residue is in the range of 30:70 to 10:90, such as in the range of 20:80 to 10:90, such as about 10:90. Alternatively, polycaprolactone, polylactide, copolymers of caprolactone and lactide or biodegradable polyurethanes can be used.

In some embodiments, the polymer to be used as reinforcing member or combined scaffold and reinforcing member of the surgical implant is substantially hydrophobic.

Particularly suitable polymers to be used as reinforcing member will be degraded more slowly than the synthetic biodegradable homogenous sheet of scaffold. Typically the suitable polymers to be used as reinforcing member will be completely degradable within 2-48 months, such as within 2-36 months, such as within 2-24 months, such as within 2-12 months of in situ application.

The scaffold may be reinforced to be easily handled by doctors in the operating room. Different methods can be used. An example of a reinforced implant according to the present invention could be a porous scaffold with welded edges and/or with a welded pattern onto the scaffold. These welded seams provide reinforcement to the scaffold and may also be used as cutting line for the surgeon if he wants to shape the scaffold to the defect.

The scaffold may be reinforced by attaching the scaffold to a non-woven membrane which may be produced by electro spinning. The membrane would preferably be significantly thinner than the scaffold. The membrane may be placed on top of the scaffold or in the centre of the scaffold.

The scaffold may alternatively be reinforced by inclusion of biodegradable thread-like sutures. The threads can either be welded or knotted in the intersections. The squares in the grid are in some embodiments at least 1 cm². In another embodiment, the threads do not have intersections, and the reinforcement comes from e.g. the “snail shaped” suture inside the scaffold. In general, the degradation time is longer for the threads than for the scaffold.

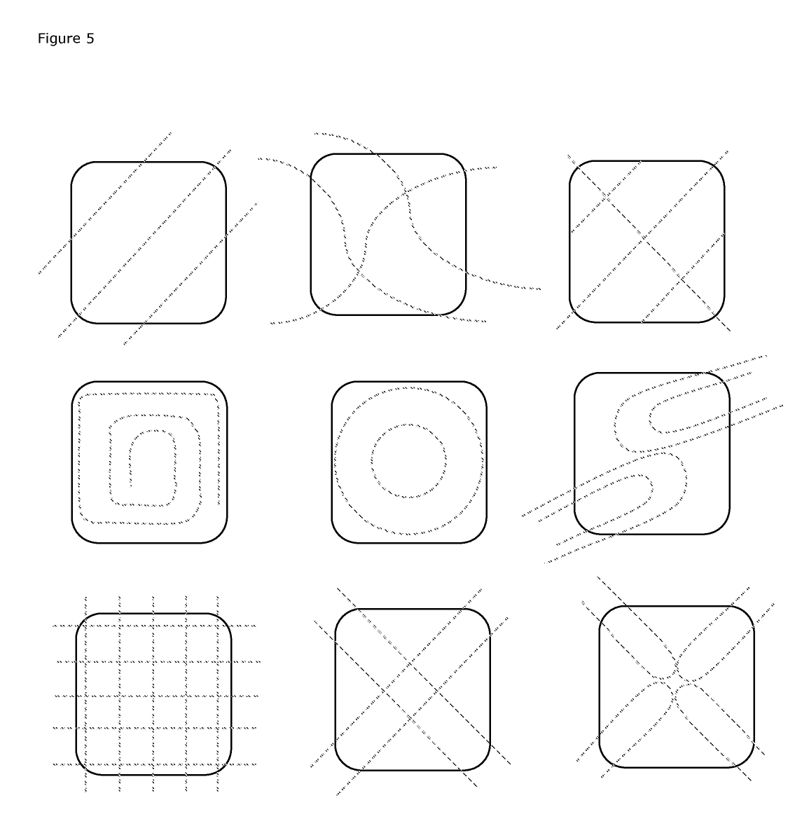

The illustrative examples of FIG. 5 depict different patterns that can be used in order to reinforce the scaffold by the inclusion of biodegradable threads.

In some particular embodiments, the reinforced implant according to the present invention is reinforced by having a combination of biodegradable threads-like sutures and welded edges and/or with a welded pattern.

Cells and Other Components that May be Applied to the Scaffold

In some embodiments according to the invention, the synthetic biodegradable scaffold is administered with a component which facilitates the cell adhesion and/or in-growth for generation of tissue within the synthetic biodegradable scaffold, such as an extracellular matrix component of any suitable tissue, such as extracellular matrix components from bladder, intestine, skin, or muscle.

In some embodiments according to the invention, the synthetic biodegradable scaffold is administered with a blood derived component and/or cells which facilitates the cell adhesion and/or in-growth for generation of tissue within the synthetic biodegradable scaffold.

A “blood derived component and/or cells”, as used herein refers to any component or cell, such as thrombocytes, leukocytes, serum proteins, etc. that may be derived from a blood sample.

Accordingly, in some embodiments according to the invention, the synthetic biodegradable scaffold is administered with a component which facilitates the cell adhesion and/or in-growth for generation of patient tissue in situ within the synthetic biodegradable scaffold, such as a component selected from the group consisting of: estrogen, estrogen derivatives, thrombin, ECM (extra cellular matrix) powder, chondroitin sulfate, hyaluronan, Hyaluronic Acid (HA), heparin sulfate, heparan sulfate, dermatan sulfate, growth factors, such as Insulin-like growth factors (IGFs), such as IGF-1 or IGF-2, or Transforming growth factors (TGFs), such as TGF-alpha or TGF-beta, or Fibroblast growth factors (FGFs), such as FGF-1 or FGF-2, or Platelet-derived growth factors (PDGFs), such as PDGF-AA, PDGF-BB or PDGF-AB, or Mechano Growth Factor (MGF), or Nerve growth factor (NGF), or Human Growth Hormone (HGH); fibrin, fibronectin, elastin, collagen, such as collagen type I and/or type II, type III, Type IV, type V and/or type VII, gelatin, and aggrecan, or any other suitable extracellular matrix component.

In one particular embodiment, hyaluronic acid is incorporated into the synthetic biodegradable scaffold. In one embodiment, the hyaluronic acid is present in the synthetic biodegradable scaffold at a proportion of between about 0.1 and about 15 wt %.

In a further specific embodiment, dermatan sulphate is incorporated into the synthetic biodegradable scaffold. In one embodiment, the dermatan sulphate is present in the synthetic biodegradable scaffold at a proportion of between about 0.1 and about 15 wt %.

The compounds discussed above which enhance cell migration and/or tissue regeneration, may be added before processing to the porous scaffold structure as pure compounds or as solutions. Alternatively they may be added, coated or encapsulated in the shape of nano or microparticles.

In some embodiments according to the invention, the synthetic biodegradable scaffold is administered with a suspension of mammalian cells or tissue, such as human stem cells or other human cells or tissue, such as muscle cells, fibroblast and endothelial cells or muscle tissue, such as cells or tissue derived from smooth, skeletal or cardiac muscle. This may usually be a suspension of muscle tissue such as biopsies or isolated muscle fibres obtained from a patient. Alternatively, it may be muscle cells or components derived from muscle cells proliferated in vitro. A muscle suspension may be applied to the surface of the scaffold before or concomitantly with the implantation. The muscle suspension used according to aspects of the invention is typically seeded with a density in the range of 1-100 mg muscle suspension per cm² of scaffold sheet. Muscle fibres are isolated from biopsies either by dissecting the muscle with e.g. scalpels or dissolution of the muscle using enzymatic treatment e.g. collagenase, to get single fibres with satellite cells.

In other embodiments according to the invention, the synthetic biodegradable scaffold is administered with a suspension of components produced by muscle cells together with these muscle cells.

In one embodiment, the suspension of mammal cells or tissue is obtained from or derived from the living individual mammal, where medical application is to be performed, i.e. is autologous.

The cells or tissue may also be homologous, i.e. compatible with the tissue to which they are applied, or may be derived from multipotent or even pluripotent stem cells, for instance in the form of allogenic cells. In one embodiment, the cells or tissue are non-autologous. In one embodiment, the cells are non-homologous. In one embodiment the cells may be allogenic, from another similar individual, or xenogenic, i.e. derived from a species other than the organism being treated. The allogenic cells could be differentiated cells, progenitor cells, or cells whether originated from multipotent (e.g. embryonic or combination of embryonic and adult specialist cell or cells, pluripotent stem cells (derived from umbilical cord blood, adult stem cells, etc.), engineered cells either by exchange, insertion or addition of genes from other cells or gene constructs, the use of transfer of the nucleus of differentiated cells into embryonic stem cells or multipotent stem cells, e.g. stem cells derived from umbilical blood cells.

In one embodiment, the method of the invention also encompasses the use of stem cells, and cells derived from stem cells, the cells may be, preferably obtained from the same species as the individual mammal being treated, such as human stem cells, or cells derived there from.

The mammalian cells used according to the invention may be supplied in the form of a cell suspension or tissue explants. Tissue explants may be directly taken from other parts of the individual mammal, and may therefore be in the form of tissue grafts such as a muscle tissue graft taken from large muscles of a mammal.

Human smooth or skeletal muscle cells, or alternatively fibroblasts and other connective tissue type cells, administered to the synthetic biodegradable scaffold would be particularly preferred. It is however envisaged that stem cells, or any other suitable precursor cells which are capable of becoming or producing muscle and/or connective cells may also be used. Typically, the cells used in this application are present in a sufficient amount of cells to result in regeneration or repair of the target tissue or defect, such as of about 0.1×10⁴ to about 10×10⁶ cells/cm², or 0.1×10⁶ cells/cm² to about 10×10⁶ cells/cm².

In some embodiments, the muscle cells used according to the invention are in the form of cell suspensions, or tissue explants.

In some embodiments, mammal cells applied to the synthetic biodegradable scaffold according to the invention, are applied in an amount of about 0.1×10⁴ cells to about 10×10⁶ cells per cm² of synthetic biodegradable scaffold.

In some embodiments, the mammal cells or tissue explants are applied to the synthetic biodegradable scaffold according to the invention at the time of medical application, such as during the surgery. It is to be understood that the surgeon may take out the tissue explants to be used according to the methods of the present invention prior to or during the surgery.

In some embodiments, mammal cells or tissue explants are cultured in the synthetic biodegradable scaffold prior to medical application, such as the surgery, for a period of at least 1 day, at least 3 days, at least 1 week, such as at least 2 weeks, such as at least 3 weeks, such as at least 6 week.

Surgical Method and the Patient

The “living individual mammal” is any living individual mammal suitable for application of the synthetic reinforced biodegradable scaffold according to the invention, and is preferably a human being, typically a patient. However, the methods of the invention may also be applicable to other mammals, such as pets including dogs, cats, and horses.

The methods for application of the synthetic biodegradable scaffold with increased strength according to the invention may be performed as, or during a method of surgery, such as a method of endoscopic, laparoscopic or other minimal invasive surgery, or conventional or open surgery.

In particular aspects of the invention, the application of the reinforced synthetic biodegradable scaffold according to the invention may be used in any medical condition requiring reconstruction surgery, wherein reinforcement at the site of surgery is required.

In particular aspects of the invention, the application of the reinforced synthetic biodegradable scaffold according to the invention is used during surgery of prolapse, such as pelvic organ prolapse, also referred to as pelvic reconstructive surgery or surgery for stress urinary incontinence.

It is to be understood that the reinforced synthetic biodegradable scaffold according to the invention may be used in reconstructive surgery involving the diaphragm, pelvic floor region or abdomen. However, it is envisaged that the reinforced synthetic biodegradable scaffold according to the invention may be used in surgical reconstructions of other fascia components of the mammal body including the dense fibrous connective tissue that interpenetrates and surrounds muscles, bones, organs, nerves and blood vessels of the body.

Accordingly, the reinforced synthetic biodegradable scaffold according to the invention may be used in the treatment of compartment syndrome, constrictive pericarditis, hemopneumothorax, hemothorax, injuries to the Dura mater, and various hernias, including preventive hernia scaffold in all abdominal surgeries.

Hernias are medical conditions for which the implant according to the present invention may be indicated. The term “hernia” as used herein includes abdominal hernias, diaphragmatic hernias and hiatal hernias (for example, paraesophageal hernia of the stomach), pelvic hernias, for example, obturator hernia, anal hernias, hernias of the nucleus pulposus of the intervertebral discs, intracranial hernias, and spigelian hernias.

The types of surgery typically associated with pelvic reconstructive surgery includes laparoscopically assisted vaginal hysterectomy, total laparoscopic hysterectomy, vaginal hysterectomy, laparoscopic vaginal vault suspension, laparoscopic sacrocolpopexy, laser vaginal rejuvenation, designer laser vaginoplasty, vaginal approach to prolapse repair incorporating mesh, sling procedures and laparoscopic paravaginal repairs.

The term “pelvic organ prolapse” as used herein refers to any medical condition involving prolapse through the pelvic wall. Other terms used and included within the definition is uterine prolapse, genital prolapse, uterovaginal prolapse, pelvic relaxation, pelvic floor dysfunction, urogenital prolapse, vaginal wall prolapse, cytocele, bladder prolapse, urethrocele, enterocele, rectocele, vaginal vault prolapse, small bowel prolapse, uterus prolapse or urethra prolapse.

One important aspect of the invention relates to a method for the treatment or for alleviating the symptoms of a connective tissue prolapse, such as pelvic organ prolapse in a living individual mammal, such as a human being, the method comprising the step of applying a synthetic biodegradable scaffold with increased strength according to the invention to the site of a defect or place requiring surgery.

As described above, another important aspect of the present invention relates to a synthetic biodegradable scaffold with reinforcing member(s) according to the invention; for use as an implant.

In one embodiment, this reinforced synthetic biodegradable scaffold according to the invention is for use in the treatment or for alleviating the symptoms of a connective tissue defect in a living individual mammal, such as a human being.

In some specific embodiments, cells that are derived from the living individual mammalian under surgery are applied to the reinforced synthetic biodegradable scaffold prior to and/or concomitantly with and/or subsequent to the application of the reinforced synthetic biodegradable scaffold to the site of defect. It is expected by the inventors of the invention that this may facilitate the uptake and tolerance of the reinforced synthetic biodegradable scaffold and increase the growth and reconstruction of tissue of the living individual mammalian at the site of surgery, and thereby increase speed of recovery for the treated mammalian, such as a human patient. In some embodiments, the cells are in a muscle suspension applied to the surface of or within the scaffold in connection with surgery and implantation of the implant according to the invention. The muscle suspension used is typically seeded with a density in the range of 1-100 mg muscle suspension per cm² of scaffold sheet. Muscle fibres may be isolated from biopsies either by dissecting the muscle with e.g. scalpels or dissolution of the muscle using enzymatic treatment e.g. collagenase, to get single fibres with satellite cells. It is to be understood that the muscle preparation may be taken for the same patient receiving the implant, i.e. an autologous preparation.

In some embodiments, the treatments according to the invention is performed as part of surgery, such as of endoscopic, laparoscopically or other minimal invasive surgery, as well as conventional or major open surgery.

In some embodiments, the treatments according to the invention is performed as part of reconstruction surgery.

The synthetic biodegradable reinforced scaffold, according to the invention, may be attached to fascia with sutures, pins, and/or various types of tissue glue. Preferably such attachment means are also biodegradable.

Kit of Parts

As described elsewhere, the present invention also provides a kit of parts, for the treatment or for alleviating the symptoms of a prolapse in a living individual mammal, the kit comprising reinforced synthetic biodegradable scaffold and instructions for use of this reinforced synthetic biodegradable scaffold.

Also provided are kits of parts for support, augmentation and regeneration of living tissue within a subject, such as in a subject with a medical prolapse, such as rectal or pelvic organ prolapse, or hernia, the kit comprising biodegradable surgical implant comprising a synthetic biodegradable scaffold and a device suitable for disintegration or disruption of a tissue sample, or alternatively a sample of autologous cells or tissue explants for use in the methods of the invention.

### Specific Embodiments of the Invention

In some embodiments of the invention, the synthetic biodegradable homogenous sheet of scaffold is hydrophilic.

In some embodiments of the invention, the synthetic biodegradable homogenous sheet of scaffold has the ability to, within 5 minutes, such as within 2 minutes at 30° C., absorb water in an amount of at least 10%, such as at least 20%, such as at least 30%, such as at least 50% of the scaffold volume.

In some embodiments, the biodegradable surgical implant, according to the invention, has a volume % of said reinforcing member less than 40%, such as less than 30%, such as less than 20%, such as less than 15%, such as less than 12% of the implant.

It has to be understood that a balance of strength, flexibility, and biodegradability of the combination of reinforcing member and scaffold material will be required depending on the specific indication being treated by the implant. Accordingly, much higher strength will be required for pelvic organ repair, than e.g. for the treatment of urinary incontinence.

In some embodiments of the invention, the synthetic biodegradable homogenous sheet of scaffold exhibit a percent elongation at break in the range of about 10-200%, such as in the range of about 30-100%, such as in the range of about 30-70%, such as in the range of about 30-60%.

In some embodiments of the invention, the surgical implant exhibit a percent elongation at break in the range of about 20-1000%, such as in the range of about 20-800%, such as in the range of about 20-500%, such as in the range of about 20-400%, such as in the range of about 20-300%.

In some embodiments of the invention, the synthetic biodegradable homogenous sheet of scaffold exhibit a tensile strength in the range of about 5-40 psi, such as in the range of about 8-30 psi, such as in the range of about 8-20 psi, such as in the range of about 8-16 psi, such as in the range of about 8-14 psi.

In some embodiments of the invention, the surgical implant exhibit a tensile strength in the range of about 300-50000 psi, such as in the range of about 500-30000 psi, such as in the range of about 1000-20000 psi, such as in the range of about 1000-10000 psi, such as in the range of about 5000-10000 psi, or in the range of about 1000-8000 psi.

In some embodiments of the invention, the scaffold material exhibit a tensile strength in the range of about 300-50000 psi, such as in the range of about 500-30000 psi, such as in the range of about 1000-20000 psi, such as in the range of about 1000-10000 psi, such as in the range of about 1000-8000 psi.

In some embodiments of the invention, the synthetic biodegradable homogenous sheet of scaffold exhibit flexibility when wetted to saturation with a liquid.

In some embodiments of the invention the synthetic biodegradable homogenous sheet of scaffold has an open pore structure with size in the range of 30-200 μm.

In some embodiments of the invention, the synthetic biodegradable homogenous sheet of scaffold mainly has vertical pore structure.

In some embodiments of the invention, the synthetic biodegradable homogenous sheet of scaffold has an open pore structure with interconnected pores.

In some embodiments of the invention, the synthetic biodegradable homogenous sheet of scaffold is prepared by freeze-drying.

In some embodiments of the invention, the biodegradable reinforcing member is based on fibres and/or threads with a thickness of about 10 nm-1000 μm, such as in the range of about 10 nm-800 μm, such as in the range of about 10 nm-500 μm.

In some embodiments of the invention, the biodegradable reinforcing member is a sheet made of woven fabric, knitted fabric, mesh, non-woven felt, made of filaments or staple fibres.

In some embodiments of the invention, the biodegradable reinforcing member is a sheet made of a woven fabric, knitted fabric, mesh, non-woven felt, made of filaments or staple fibres, wherein the sheet has a thickness of 30 μm-5 mm, such a 3-5 mm, such as 1-4 mm.

In some embodiments of the invention, the synthetic biodegradable homogenous sheet of scaffold is completely degradable within 1-48 months, such as 4-36, such as 6-24, or 1-12 months of in situ application.

In some embodiments of the invention, the biodegradable reinforcing member promotes cell attachment and in-growth of cells derived from the living tissue in said subject or from the application of cell or tissue explants.

In some embodiments of the invention, the reinforcing biodegradable member is completely degradable within 1-12 months, such as in the range of 2-12 months of in situ application.

In some embodiments of the invention, the reinforced biodegradable member is made from a polymer of poly(lactide-co-glycolide) PLGA, such as a polymer, wherein the molar ratio of (i) lactide units and (ii) glycolide units in the poly(lactide-co-glycolide) residue is in the range of 90:10 to 10:90, such as in the range of 80:20 to 10:90, such as about 10:90.

In some embodiments of the invention, the synthetic biodegradable homogenous sheet of scaffold is a polymer of the general formula:

A-O—(CHR¹CHR²O)ₙ—B
wherein;
A is a poly(lactide-co-glycolide) residue of a molecular weight of at least 4000 g/mol, the molar ratio of (i) lactide units and (ii) glycolide units in the poly(lactide-co-glycolide) residue being in the range of 80:20 to 10:90;
B is either a poly(lactide-co-glycolide) residue as defined for A or is selected from the group consisting of hydrogen, C₁₋₆-alkyl and hydroxy protecting groups, one of R¹ and R² within each —(CHR¹CHR²O)— unit is selected from hydrogen and methyl, and the other of R¹ and R² within the same —(CHR¹CHR²O)— unit is hydrogen;
n represents the average number of —(CHR¹CHR²O)— units within a polymer chain and is an integer in the range of 10-1000; and wherein
the molar ratio of (iii) polyalkylene glycol units —(CHR¹CHR²O)— to the combined amount of (i) lactide units and (ii) glycolide units in the poly(lactide-co-glycolide) residue(s) is at the most 20:80;
and wherein the molecular weight of the copolymer is at least 10,000 g/mol, preferably at least 15,000 g/mol.

In some embodiments of the invention, both of R1 and R2 within each unit are hydrogen.

In some embodiments of the invention, B is a poly(lactide-co-glycolide) residue as defined for A.

In some embodiments of the invention, B is C1-6-alkyl.

In some embodiments of the invention, B is a hydroxy protecting group.

In some embodiments of the invention, B is a hydroxy group.

In some embodiments of the invention, the weight percentage of (iii) polyalkylene glycol units —(CHR1CHR2O)— to the combined amount of (i) lactide units and (ii) glycolide units in the poly(lactide-co-glycolide) residue(s) is in the range of 4%-10% w/w.

In some embodiments of the invention, the synthetic biodegradable homogenous sheet of scaffold is prepared by freeze-drying a solution comprising the biodegradable polymer in solution.

In some embodiments of the invention, the reinforcing member is made of biodegradable fibres and/or threads.

In some embodiments of the invention, the reinforcing member is in a pattern selected from the group consisting of: triangles, circles, connecting waves, non-connecting waves, and overlapping waves.

In some embodiments of the invention, the reinforcing member is made from welding seams of the synthetic biodegradable homogenous sheet of scaffold, such as welding seams provided in square- and hexagonal pattern or along the edge of the implant.

In some embodiments of the invention, the synthetic biodegradable homogenous sheet of scaffold is a polymer of molecular weight greater than about 1 kDa, such as between about 1 kDa and about 1,000,000 kDa, such as between 25 kDa and 100 kDa.

In some embodiments of the invention, the implant further comprises within the scaffold one or more components which facilitate the cell adhesion and/or in-growth for regeneration of tissue, such as a component selected from the group consisting of: estrogen, estrogen derivatives, thrombin, ECM powder, chondroitin sulfate, hyaluronan, hyaluronic acid (HA), heparin sulfate, heparan sulfate, dermatan sulfate, growth factors, fibrin, fibronectin, elastin, collagen, such as collagen type I and/or type II, gelatin, and aggrecan, or any other suitable extracellular matrix component.

In some embodiments of the invention, the implant comprises within the scaffold one or more components selected from the group consisting of growth factors, such as Insulin-like growth factors (IGFs), such as IGF-1 or IGF-2, or Transforming growth factors (TGFs), such as TGF-alpha or TGF-beta, or Fibroblast growth factors (FGFs), such as FGF-1 or FGF-2, or Platelet-derived growth factors (PDGFs), such as PDGF-AA, PDGF-BB or PDGF-AB, or Nerve growth factor (NGF), or Human growth hormone (hGH), and Mechano Growth Factor (MGF).

In some embodiments of the invention the implant comprises within said scaffold a sample of cells or tissue explants.

In some embodiments of the invention, the implant is formed as a tube and/or comprises a flap and/or pocket suitable for application of a suspension of a sample of cells or tissue explants to the implant.

In some embodiments of the invention, the implant comprises two or more separated pieces of synthetic biodegradable homogenous sheets of scaffold, such as 3, 4, 5 or 6 pieces of synthetic biodegradable homogenous sheets of scaffold attached to a reinforcing member, such as a mesh of a different polymer.

In some embodiments of the invention, the implant comprises two or more, such as 4 or 6 arms or extensions for attachment to structures in the site of implantation, such as in the pelvic region.

In some embodiments of the method, according to invention, the subject is suffering from a medical prolapse, such as pelvic organ prolapse or hernia.

In some embodiments of the method, according to the invention, the method comprises implantation of said biodegradable surgical implant together with a sample of cells or tissue explants within said subject at the site of implantation.

In some embodiments the sample of cells or tissue explants are taken from the patient in the operating room and placed on the implant at the site of implantation during surgery. Alternatively, the cells or tissue explants to be used together with the implant have been taken from the patient prior to surgery. In another alternative, the implant and cells or tissue explants are provided as a kit and used together during surgery.

In some embodiments of the methods, according to the invention, the cells or tissue explants are autologous, homologus (allogenic) or xenogenic in origin relative to cells of said living tissue in a subject. In some embodiments of the method according to the invention, the cells or tissue explants are autologous to the subject having the implant.

In some embodiments of the methods, according to the invention, the synthetic biodegradable scaffold is a homogenous sheet.

In some embodiments, the biodegradable surgical implant is, according to the invention, used in the methods of the invention.

In some embodiments of the methods, according to the invention, the subject is suffering from a medical prolapse, such as pelvic organ prolapse, or hernia, or urinary incontinence.

In some embodiments of the methods, according to the invention, the amount of cells in said sample of cells or tissue explants used is in the range of about 0.1×10⁴ cells to about 10×10⁶ cells per cm² of implant.

In some embodiments of the methods, according to the invention, the tissue explants is from muscle tissue, stem cells, such as stem cells capable of differentiation into myoblasts, or fibroblasts; or combinations thereof.

In some embodiments of the methods, according to the invention, the cells or tissue explants are derived from a human.

In some embodiments of the methods, according to the invention, the cells or tissue explants are cultured in vitro for a certain amount of time on or within said synthetic biodegradable homogenous sheet of scaffold prior to implantation.

In some embodiments of the methods, according to the invention, the cells or tissue explants are not cultured in vitro prior to implantation.

In some embodiments of the methods, according to the invention, the cells or tissue explants are harvested and used according to the method in the operating room.

In some embodiments of the methods, according to the invention, the method further comprises application to said biodegradable surgical implant of a composition comprising a component which facilitates the cell adhesion and/or in-growth for regeneration of tissue, such as a component selected from the group consisting of: estrogen, estrogen derivatives, thrombin, ECM powder, chondroitin sulfate, hyaluronan, hyaluronic acid (HA), heparin sulfate, heparan sulfate, dermatan sulfate, growth factors, fibrin, fibronectin, elastin, collagen, such as collagen type I and/or type II, gelatin, and aggrecan, or any other suitable extracellular matrix component.

In some embodiments of the methods, according to the invention, the method further comprises application to said biodegradable surgical implant of a composition comprising a component selected from the group consisting of growth factors, such as Insulin-like growth factors (IGFs), such as IGF-1 or IGF-2, or Transforming growth factors (TGFs), such as TGF-alpha or TGF-beta, or Fibroblast growth factors (FGFs), such as FGF-1 or FGF-2, or Platelet-derived growth factors (PDGFs), such as PDGF-AA, PDGF-BB or PDGF-AB, or Nerve growth factor (NGF), or Human growth hormone (hGH), and Mechano Growth Factor (MGF).

In some embodiments the kits, according to the present invention, comprise a device suitable for disintegration or disruption, which device comprises holes or a mesh for crushing said tissue sample by the application of pressure by which the tissue sample is forced through said mesh or holes.

In some embodiments the kits, according to the present invention, comprise a device suitable for disintegration or disruption based on a mill, ultra sonic treatment, high pressure, or physical force from knives or other instruments, one example being a homogenizer with rotating knives.

### Numbered Embodiments of the Invention

1. A biodegradable surgical implant for support, augmentation and regeneration of living tissue in a subject, comprising
   a) a synthetic biodegradable homogenous sheet of scaffold,
   b) one or more biodegradable reinforcing member;
characterised in that said synthetic biodegradable homogenous sheet of scaffold being hydrophilic.
2. The biodegradable surgical implant according to embodiment 1, wherein said synthetic biodegradable homogenous sheet of scaffold has the ability to, within 5 minutes, such as within 2 minutes at 30° C., absorb water in an amount of at least 10%, such as at least 20%, such as at least 30%, such as at least 50% of the scaffold volume.
3. The biodegradable surgical implant according to any one of embodiments 1 or 2, wherein the volume % of said reinforcing member is less than 40% of the implant.
4. The biodegradable surgical implant according to any one of embodiments 1-3, wherein said synthetic biodegradable homogenous sheet of scaffold exhibit a percent elongation at break in the range of about 10-200%, such as in the range of about 30-100%, such as in the range of about 30-70%, such as in the range of about 30-60%.
5. The biodegradable surgical implant according to any one of embodiments 1-4, wherein said surgical implant exhibit a percent elongation at break in the range of about 20-1000%, such as in the range of about 20-800%, such as in the range of about 20-500%, such as in the range of about 20-400%, such as in the range of about 20-300%.
6. The biodegradable surgical implant according to any one of embodiments 1-5, wherein said synthetic biodegradable homogenous sheet of scaffold exhibit a tensile strength in the range of about 5-40 psi, such as in the range of about 8-30 psi, such as in the range of about 8-20 psi, such as in the range of about 8-16 psi, such as in the range of about 8-14 psi.
7. The biodegradable surgical implant according to any one of embodiments 1-6, wherein said surgical implant exhibit a tensile strength in the range of about 300-50000 psi, such as in the range of about 500-30000 psi, such as in the range of about 1000-20000 psi, such as in the range of about 1000-10000 psi, such as in the range of about 1000-8000 psi.
8. The biodegradable surgical implant according to any one of embodiments 1-7, wherein said synthetic biodegradable homogenous sheet of scaffold exhibit flexibility when wetted to saturation with a liquid.
9. The biodegradable surgical implant according to any one of embodiments 1-8, wherein said synthetic biodegradable homogenous sheet of scaffold has an open pore structure with size in the range of 30-200 μm.
10. The biodegradable surgical implant according to any one of embodiments 1-9, wherein said synthetic biodegradable homogenous sheet of scaffold has mainly vertical pore structure.
11. The biodegradable surgical implant according to any one of embodiments 1-10, wherein said synthetic biodegradable homogenous sheet of scaffold has an open pore structure with interconnected pores.
12. The biodegradable surgical implant according to any one of embodiments 1-11, wherein said synthetic biodegradable homogenous sheet of scaffold is prepared by freeze-drying.
13. The biodegradable surgical implant according to any one of embodiments 1-12, wherein said biodegradable reinforcing member is based on fibres and/or threads with a thickness of about 10 nm-1000 μm, such as in the range of about 10 nm-800 μm, such as in the range of about 10 nm-500 μm.
14. The biodegradable surgical implant according to any one of embodiments 1-13, wherein said biodegradable reinforcing member is a sheet made of a woven fabric, knitted fabric, mesh, non-woven felt, made of filaments or fibres.
15. The biodegradable surgical implant according to embodiment 14, wherein said sheet has a thickness of 30 μm-5 mm, such a 3-5 mm, such as 1-4 mm.
16. The biodegradable surgical implant according to any one of embodiments 1-15, wherein said synthetic biodegradable homogenous sheet of scaffold is completely degradable within 1-12 months of in situ application.
17. The biodegradable surgical implant according to any one of embodiments 1-16, wherein said biodegradable reinforcing member promotes cell attachment and in-growth of cells derived from the living tissue in said subject or from the application of cell or tissue explants.
18. The biodegradable surgical implant according to any one of embodiments 1-17, wherein said reinforcing biodegradable member is completely degradable within 1-12 months of in situ application.
19. The biodegradable surgical implant according to any one of embodiments 1-18, wherein said reinforced biodegradable member is made from a polymer of poly(lactide-co-glycolide) PLGA, such as a polymer wherein the molar ratio of (i) lactide units and (ii) glycolide units in the poly(lactide-co-glycolide) residue is in the range of 90:10 to 10:90, such as in the range of 80:20 to 10:90, such as about 10:90.
20. The biodegradable surgical implant according to any one of embodiments 1-19, wherein said synthetic biodegradable homogenous sheet of scaffold is a polymer of the general formula:

A-O—(CHR¹CHR²O)ₙ—B
wherein;
A is a poly(lactide-co-glycolide) residue of a molecular weight of at least 4000 g/mol, the molar ratio of (i) lactide units and (ii) glycolide units in the poly(lactide-co-glycolide) residue being in the range of 80:20 to 10:90;
B is either a poly(lactide-co-glycolide) residue as defined for A or is selected from the group consisting of hydrogen, C₁₋₆-alkyl and hydroxy protecting groups, one of R¹ and R² within each —(CHR¹CHR²O)— unit is selected from hydrogen and methyl, and the other of R¹ and R² within the same —(CHR¹CHR²O)— unit is hydrogen;
n represents the average number of —(CHR¹CHR²O)— units within a polymer chain and is an integer in the range of 10-1000; and wherein
the molar ratio of (iii) polyalkylene glycol units —(CHR¹CHR²O)— to the combined amount of (i) lactide units and (ii) glycolide units in the poly(lactide-co-glycolide) residue(s) is at the most 20:80;
and wherein the molecular weight of the copolymer is at least 10,000 g/mol, preferably at least 15,000 g/mol.
21. The biodegradable surgical implant according to embodiment 20, wherein both of R1 and R2 within each unit are hydrogen.
22. The biodegradable surgical implant according to embodiment 20 or 21, wherein B is a poly(lactide-co-glycolide) residue as defined for A.
23. The biodegradable surgical implant according to any one of embodiments 20-22, wherein B is C1-6-alkyl.
24. The biodegradable surgical implant according to any one of embodiments 20-23, wherein B is a hydroxy protecting group.
25. The biodegradable surgical implant according to any one of embodiments 20-23, wherein B is a hydroxy group.
26. The biodegradable surgical implant according to any one of embodiments 20-25, wherein the weight percentage of (iii) polyalkylene glycol units —(CHR¹CHR²O)— to the combined amount of (i) lactide units and (ii) glycolide units in the poly(lactide-co-glycolide) residue(s) is in the range of 4%-10% w/w.
27. The biodegradable surgical implant according to any one of embodiments 1-26, wherein said synthetic biodegradable homogenous sheet of scaffold is prepared by freeze-drying a solution comprising the biodegradable polymer in solution.
28. The biodegradable surgical implant according to any one of embodiments 1-27, wherein said reinforcing member is made of biodegradable sutures and/or threads.
29. The biodegradable surgical implant according to embodiment 28, wherein said reinforcing member is in a pattern selected from the group consisting of: triangles, circles, connecting waves, non-connecting waves, and overlapping waves.
30. The biodegradable surgical implant according to any one of embodiments 1-29, wherein said reinforcing member is made from welding seams of the synthetic biodegradable homogenous sheet of scaffold, such as welding seams provided in square- and hexagonal pattern or along the edge of the implant.
31. The biodegradable surgical implant according to any one of embodiments 1-30, wherein said synthetic biodegradable homogenous sheet of scaffold is a polymer of molecular weight greater than about 1 kDa, such as between about 1 kDa and about 1,000,000 kDa, such as between 25 kDa and 100 kDa.
32. The biodegradable surgical implant according to any one of embodiments 1-31, which implant further comprises, within said scaffold, one or more components which facilitate the cell adhesion and/or in-growth for regeneration of tissue, such as a component selected from the group consisting of: estrogen, estrogen derivatives, thrombin, ECM powder, chondroitin sulfate, hyaluronan, hyaluronic acid (HA), heparin sulfate, heparan sulfate, dermatan sulfate, growth factors, fibrin, fibronectin, elastin, collagen, such as collagen type I and/or type II, gelatin, and aggrecan, or any other suitable extracellular matrix component.
33. The biodegradable surgical implant according to any one of embodiments 1-32, which implant further comprises, within said scaffold, one or more components selected from the group consisting of growth factors, such as Insulin-like growth factors (IGFs), such as IGF-1 or IGF-2, or Transforming growth factors (TGFs), such as TGF-alpha or TGF-beta, or Fibroblast growth factors (FGFs), such as FGF-1 or FGF-2, or Platelet-derived growth factors (PDGFs), such as PDGF-AA, PDGF-BB or PDGF-AB, or Nerve growth factor (NGF), or Human growth hormone (hGH), and Mechano Growth Factor (MGF).
34. The biodegradable surgical implant according to any one of embodiments 1-33, which implant further comprises, within said scaffold, a sample of cells or tissue explants.
35. A method for support, augmentation and regeneration of living tissue within a subject with a medical prolapse, such as pelvic organ prolapse, or hernia, said method comprising implantation of a biodegradable surgical implant comprising a synthetic biodegradable homogenous sheet of scaffold together with a sample of cells or tissue explants within said subject at the site of said prolapse or hernia.
36. A method for support, augmentation and regeneration of living tissue within a subject, said method comprising implantation of biodegradable surgical implant according to any one of embodiments 1-34 within said subject.
37. The method according to embodiment 36, wherein said subject is suffering from a medical prolapse, such as pelvic organ prolapse or hernia.
38. The method according to embodiments 36 or 37, wherein said method comprises implantation of said biodegradable surgical implant together with a sample of cells or tissue explants within said subject at the site of implantation.
39. The method according to embodiments 35 or 38, wherein said cells or tissue explants are autologous, homologus (allogenic) or xenogenic in origin relative to cells of said living tissue in a subject.
40. The method according to any one of embodiments 35, or 38-39, wherein the amount of cells in said sample of cells or tissue explants used, is in the range of about 0.1×10⁴ cells to about 10×10⁶ cells per cm² of implant.
41. The method according to any one of embodiments 35, or 38-40, wherein the tissue explants is from muscle tissue, stem cells, such as stem cells capable of differentiation into myoblasts, or fibroblasts; or combinations thereof.
42. The method according to any one of embodiments 35, or 38-41, wherein said cells or tissue explants are derived from a human.
43. The method according to any one of embodiments 35, or 38-42, wherein said cells or tissue explants are cultured in vitro for a certain amount of time on or within said synthetic biodegradable homogenous sheets of scaffold prior to implantation.
44. The method according to any one of embodiments 35-43, which method further comprises application to said biodegradable surgical implant of a composition comprising a component which facilitates the cell adhesion and/or in-growth for regeneration of tissue, such as a component selected from the group consisting of: estrogen, estrogen derivatives, thrombin, ECM powder, chondroitin sulfate, hyaluronan, hyaluronic acid (HA), heparin sulfate, heparan sulfate, dermatan sulfate, growth factors, fibrin, fibronectin, elastin, collagen, such as collagen type I and/or type II, gelatin, and aggrecan, or any other suitable extracellular matrix component.
45. The method according to any one of embodiments 35-44, which method further comprises application to said biodegradable surgical implant of a composition comprising a component selected from the group consisting of growth factors, such as Insulin-like growth factors (IGFs), such as IGF-1 or IGF-2, or Transforming growth factors (TGFs), such as TGF-alpha or TGF-beta, or Fibroblast growth factors (FGFs), such as FGF-1 or FGF-2, or Platelet-derived growth factors (PDGFs), such as PDGF-AA, PDGF-BB or PDGF-AB, or Nerve growth factor (NGF), or Human growth hormone (hGH), and Mechano Growth Factor (MGF).
46. A method for the preparation of a biodegradable surgical implant according to any one of embodiments 1-34, which method comprises the simultaneous of sequential steps of
   a) preparing said synthetic biodegradable homogenous sheets of scaffold;
   b) preparing and incorporating said one or more biodegradable reinforcing member within said synthetic biodegradable homogenous sheets of scaffold;
   c) optionally incorporating one or more components as defined in any one of embodiments 32-34.
47. A kit comprising
   a) a biodegradable surgical implant according to any one of embodiments 1-34;
   b) a sample of cells or tissue explants; and
   c) optionally instructions for use in a method for support, augmentation and regeneration of living tissue within a subject with a medical prolapse, such as rectal or pelvic organ prolapse, or hernia, said method comprising implantation of said biodegradable surgical implant together with a sample of cells or tissue explants within said subject at the site of said prolapse or hernia.
48. An implant according to any one of the embodiments 1-34 for use as a medicament.
49. An implant according to any one of the embodiments 1-34 for use in the treatment of a disease related to pelvic organ prolapse and hernia.

### EXAMPLES

### Example 1

Welding Seams

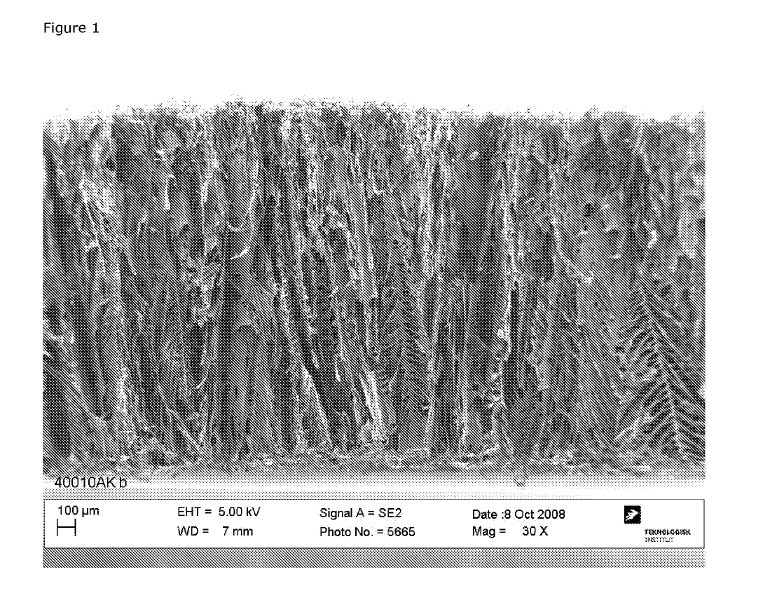

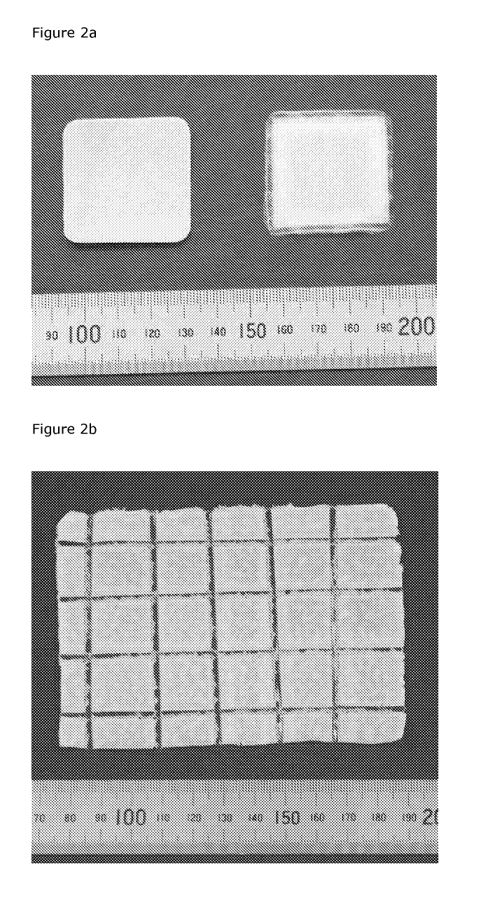

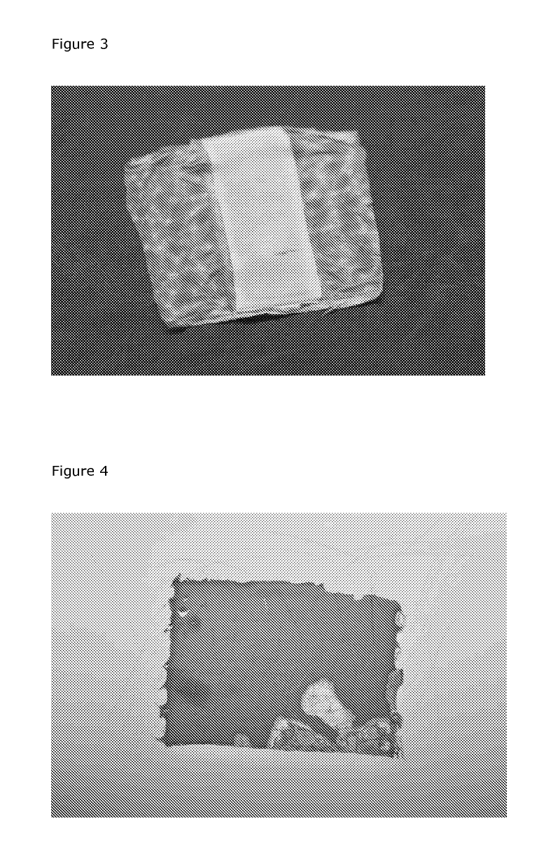

Scaffolds are sheets of freeze-dried structures. They are made by freezing/freeze-drying a solution of polymer. This results in a porous open celled structure where the pores are oriented mainly along the direction of freezing. This orientation can be seen on FIG. 1.

This orientation means that the material has very low tear strength. To strengthen the material, weld seams are added. The material is compressed/melted, thereby loosing the structure described above and gaining strength. This means that the material can take the stress of suturing and handling. This welding can be done by pulse welding, laser welding or similar heat treatment.

These welding seams can be either added only to the edge or in a grid pattern for even more strength. By having a grid pattern, it will be possible to cut the scaffold to size without losing the strength.

2 Layer Scaffold with Different Degradation Times

It can be desirable to have a layer of the device that supports cell growth and degrades rapidly and a layer that stays longer for support and strength. The scaffold can be a 2-layer structure made either by casting/freeze-drying a 2 layer structure and subsequently welding this for strength, or welding the slower degrading layer in a grid pattern and subsequently attaching the faster degrading layer onto this (e.g. by welding). The combination of polymers that will give a fast/slow degrading composite is known to persons skilled in the art. It can also be made by welding a faster degrading scaffold to a backing of a slower degrading mesh.

Mounting Scaffold on a Backing Layer

Another way to strengthen the scaffold is to attach the scaffold to a stronger backing material.

The backing material can be a non-woven biodegradable fibre material, e.g. a mat of electrospun biodegradable polyester. The scaffold material is made of a material that degrades rapidly in the body (8 weeks). It can be desirable to have a backing material with a longer degradation time e.g. 6 months or longer. Examples of materials with a longer degradation time is PLGA with a glycolide content >50 mole %, PLGA with a lactide content >50 mole %, poly(D,L-lactide), poly(L-lactide), poly(caprolactone), poly(3-hydroxybutyrate). Other suitable materials that may be used are easily selected and used by the person skilled in the art.

The scaffold can be fixed to the backing by welding.

Reinforcement with Threads

By incorporation of a grid of biodegradable suture it is possible to reinforce the scaffold structure.

### EXPERIMENTAL

Scaffolds

MPEG-PLGA 2-30 kDa with a 50:50 molar G:L-ratio is dissolved to 4% w/v in dioxane. The bottom of a 10×10 cm alu-mould is covered with dioxane and the mould is cooled to −5° C. When the dioxane is frozen, 27 mL of polymer solution is cast on top of the frozen layer, and the mould is again cooled to −5° C. The frozen polymer solution is then freeze-dried, and the freeze-dried structure is stored for 5 days in a vacuum desiccator.

2-Layer Scaffold with 2 Different Degradation Times

MPEG-PLGA 2-30 kDa with a 50:50 molar G:L-ratio is dissolved to 4% w/v in dioxane.

PDLLA is dissolved to 4% w/v in dioxane.

The bottom of a 10×10 cm alu-mould is covered with dioxane and the mould is cooled to −5° C. When the dioxane is frozen, 13.5 mL of PDLLA solution is cast on top of the frozen layer, and the mould is again cooled to −5° C. When frozen, 13.5 mL of MPEG-PLGA solution is cast on top of the frozen layer, and the mould is again cooled to −5° C. The frozen bi-layer structure is then freeze-dried. The scaffold now consists of 2 layers with different degradation times. The MPEG-PLGA layer will degrade in ˜8 weeks (in-vivo) and the PDLLA-layer will degrade in ˜12 months (in-vivo)

Welding

A HAWO hpl450 pulse welding device set at sealing time 3, cooling time 7. The welding seam is 2 mm wide

Electrospinning

2.5 g PLGA 10:90 (PURAC purasorb PLG® 1017) is dissolved to 25 mL in hexafluoroisopropanol and electrospun to sheets.

6 g PDLLA (Phusis) is dissolved in 20 g acetone and electrospun (1 kV/cm) to sheets.

### Example 2

Flexibility

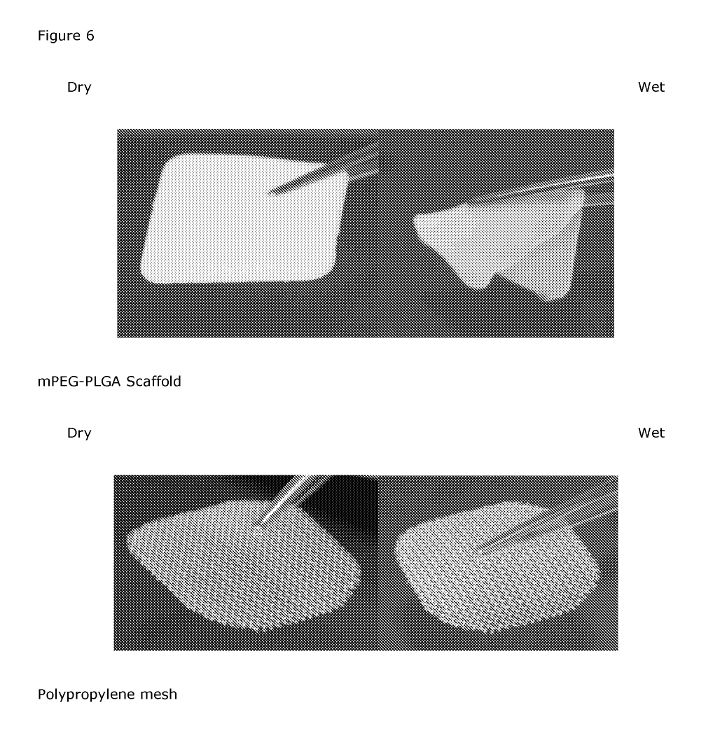

Regarding the flexibility of the scaffold, as it is depicted in FIG. 6 that when the scaffold is dry, it is rigid. On the other hand, once it is wet it becomes very pliable. This compared to the polypropylene mesh, which does not become less rigid, after exposure to water.

### Example 3

Determination of the Strength of the Scaffolds with and without Weld Seams

Apparatus: Lloyd tensile tester with a 50 N load cell. Speed: 100 mm/min, separation of jaws 20 mm.

Scaffolds (40×40×2 mm) are cut into strips that are 5 mm wide. In some of these, a 3 mm weld seam is made along the length of the strip (this weld seam has a thickness of approximately 0.1 mm). The maximum force and elongation at break is measured for both unmodified and welded strips.

| | Maximum | Deflection at | % elongation | | |
|---|---|---|---|---|---|
| | Load (N) | Break (mm) | at break | N/m2 | psi |
| Unmodified | 0.91 | 10.28 | 51.39 | 9.13E+04 | 13 |
| Unmodified | 0.92 | 10.60 | 53.02 | 9.16E+04 | 13 |
| Unmodified | 0.70 | 8.74 | 43.71 | 6.99E+04 | 10 |
| Unmodified | 0.84 | 11.09 | 55.46 | 8.37E+04 | 12 |
| welded | 15.05 | 37.81 | 189.07 | 5.02E+07 | 7275 |
| welded | 13.51 | 59.49 | 297.44 | 4.50E+07 | 6533 |
| welded | 9.53 | 41.31 | 206.54 | 3.18E+07 | 4607 |
| welded | 11.19 | 44.28 | 221.38 | 3.73E+07 | 5409 |

### Example 4

Use of Muscle Biopsies in the Implant Comprising the Scaffold

A muscle biopsy is placed in a container with an appropriate buffer e.g. cell media, PBS, etc. Cells and muscle fibres are isolated from the biopsies by the use of a tissue grinder (e.g. Sigma-Aldrich). The muscle suspension is afterwards applied to the surface of the scaffold before implantation.

In one set of experiments, muscle fibres are isolated from biopsies either by dissecting the muscle with e.g. scalpels or dissolution of the muscle using enzymatic treatment e.g. collagenase, to get single fibres with satellite cells. These fibres are applied to the surface of the scaffold before implantation.

In another set of experiments, tissue explants from muscle tissue are from muscle dissected into a muscle puree by e.g. scalpels or wherein muscle fibres are isolated from the remaining tissue using mechanically or enzymatic methods, or wherein the muscle tissue is grinded into a muscle slurry, all of which comprises a population of fibroblasts, muscle fibres and muscle precursor cells like satellite cells and myoblasts.

### Example 5

Methods for Crushing Tissue

In some embodiments according to the invention, in vitro grown cells may be seeded on the device comprising a scaffold before implantation. Cells for this purpose may be provided by taking a biopsy and extract and expand the cells in vitro before implantation. However, this procedure is expensive and may have regulatory issues.

Instead, a tissue puree (containing cells) made directly in the operating room may be applied with the device as described in the following.

The operating principle is that tissue from a biopsy is forced through a screen mesh with pressure. This crushes the sample into a mush that can be applied to the scaffold before implantation.

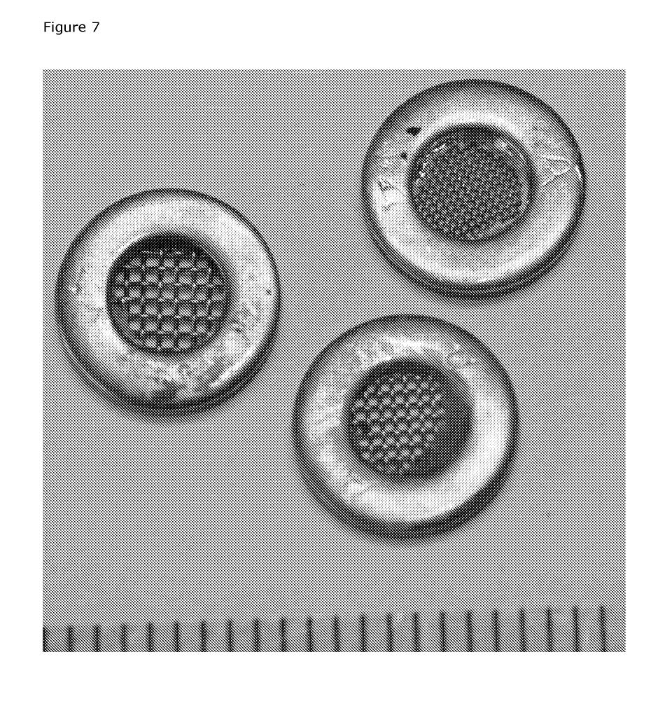

The mesh may be circular and may have reinforcement around the edge as seen in FIG. 7.

The mesh may be loaded into a plastic syringe, which is then loaded with tissue before applying pressure.

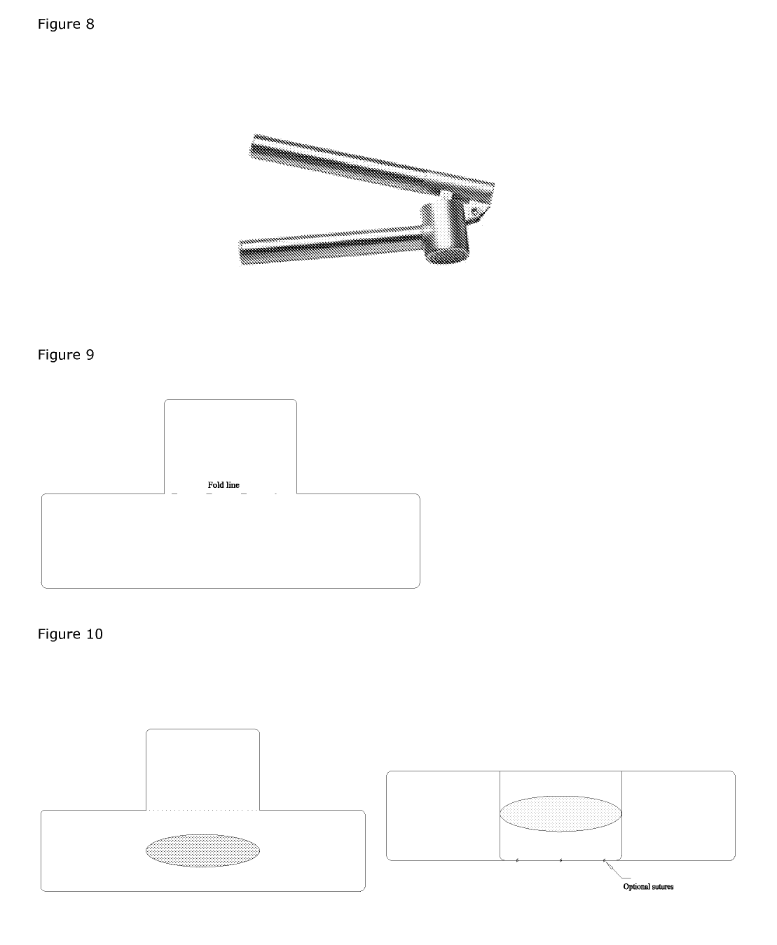

However, devices where higher pressure is applied may be used with advantage. Accordingly, a metal piston in a metal cylinder as shown in FIG. 8 may be used.

Alternatively, the biopsy can be crushed with commercial tissue grinder or a homogenizer with rotating knives.

### Example 6

The biodegradable surgical implant comprising a scaffold, and in particular the implant used for pelvic organ repair, used according to the present invention is designed to facilitate the application of cells before implantation. In addition to the shapes shown in the following example, the implant may be shaped to fit in the pelvic region.

All designs may be reinforced with additional weld seams at the edges as reinforcement and anchoring points for sutures when attaching the device to the pelvic floor.

1. The Flap:

A rectangular sheet of non-woven material with a flap attached:

The flap can be either:

a) The same non-woven material and same thickness. In this case the device can be cut from a single sheet of material. The fold line might need to be partially cut or embossed.

b) The same material and different thickness. In this case, the flap has to be attached to the rectangle by suitable means (preferably welding).

c) A different material but still non-woven and any thickness. Again, the flap has to be attached.

d) A different material and a different process (e.g. freeze-dried). The flap is attached by welding.

The cells are applied, the flap is folded on top and the construct is optionally closed by e.g. sutures as shown below.

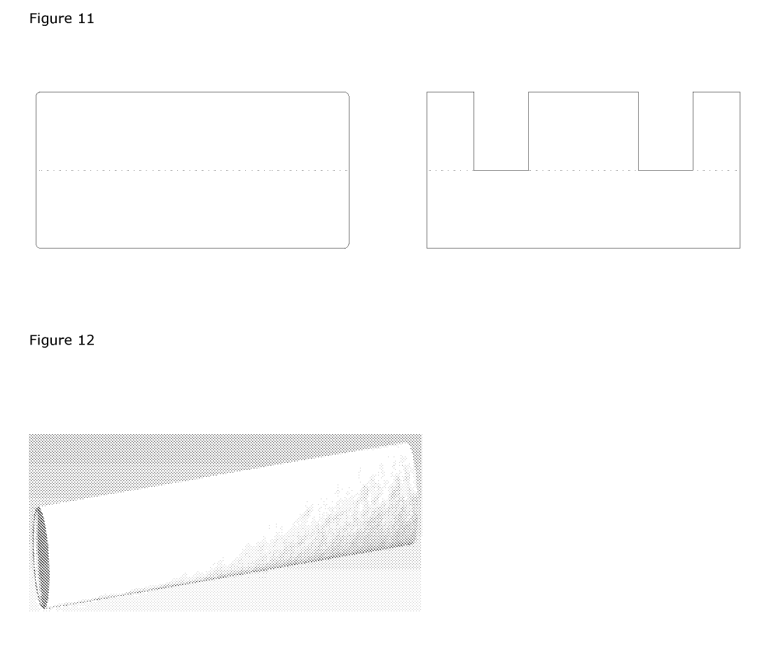

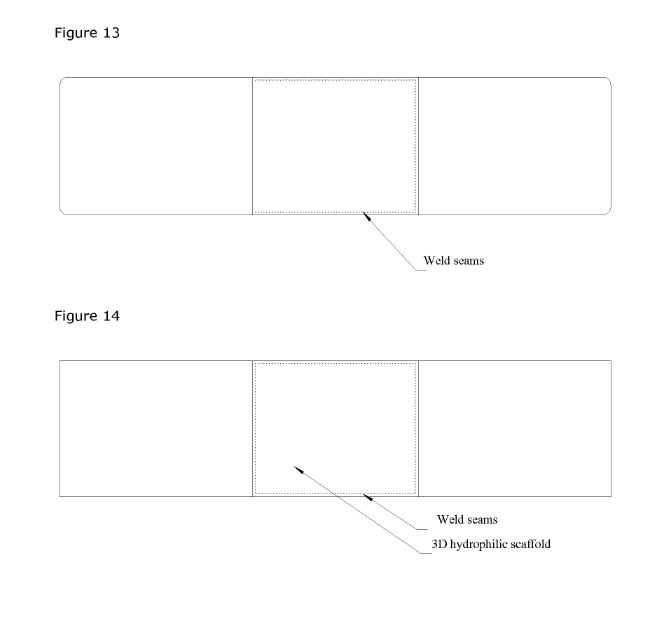

The flap can be either partial (FIG. 9), full-length (FIG. 11 left) or segmented (FIG. 11 right):

2. The Tube

A tube, either seamless, or with seams.

Variations for the tube resemble those for the flap:

a) Single material, one thickness. This can be either seamless or made by a single weld in a rectangular sheet.

b) Single material, one or two thicknesses. Two rectangles of different or same thickness joined by two welds.

c) Two materials, one or two thicknesses. Two rectangles of different materials and of different or same thickness joined by two welds.

The cells are inserted into the tube and the tube is flattened and implanted. No closure step necessary.

3. The Pocket

A variation of the tube and the flap. A rectangle is welded to the device with 3 seams. All the possible variations for the flap and the tube apply for the pockets as well.

4. Absorbent 3D-Scaffold

A 3D hydrophilic scaffold is welded to a mesh. The cells are applied to and taken up by the scaffold. This design can be considered to be a sub-group of the other designs. The design can incorporate one scaffold (as in FIG. 14) fixed to a mesh, or 2 or more scaffolds fixed to a mesh.

5. The Plain Sheet

A simple rectangular sheet. Special tricks might be needed to facilitate wetting of and/or attachment of cells to the sheet:

1) Glue (e.g. fibrin) to adhere the cells.

2) Modification of fibres to facilitate wetting.
a) Coaxial spinning with an outer layer of a hydrophilic polymer.
b) Coating with hydrophilic polymer.
c) Co-spinning 2 different fibres, one of them hydrophilic:
   i) Mix of fibres.
   ii) Layers, one hydrophilic, one hydrophobic.
   iii) Gradient starting with hydrophobic and ending with hydrophilic.
   iv) All combinations of i, ii, and iii.

3) Any combination of features of 1 and/or 2.

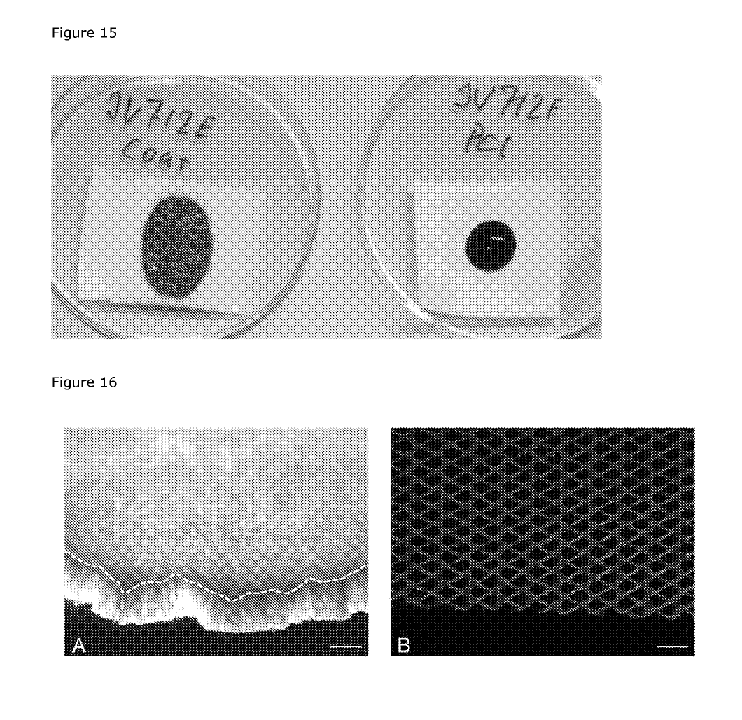

An example of a sheet modified to facilitate wetting is shown in FIG. 15. Poly(ε-caprolactone) is a hydrophobic polymer, but by coating the fibre with a small amount (˜3%) of a hydrophilic polymer (MPEG-PLGA 2-30 50DL), wetting with blood is faster.

All designs in examples 1-5 can further have arms/extensions for fixing the scaffold to structures in the pelvic region as seen in FIG. 19.

### Example 7

Use of Implant for Pelvic Reconstructive Surgery

A resorbable implant consisting of MPEG-PLGA (methoxypolyethyleneglycol-poly(lactic-co-glycolic acid)) used. It is freeze-dried and made more hydrophilic to promote in-growth of cells and improve the repairing process. FIG. 16 illustrates the structure.

The aim of the study was to investigate biocompatibility and durability of three MPEG-PLGA implants: plain, enriched with extracellular matrix (ECM, ACell, Inc.) or estrogen (Estradiol, Sigma-Aldrich, Inc.).

Study Design, Materials and Methods

Twenty implants of each preparation, sized 1×2 cm, were implanted subcutaneously on the abdomen of rats, two in each. As a control, a sham site with blunt dissection and a single stitch of Vicryl suture was used. Explantation was carried out after 3 weeks (15 rats) and after 8 weeks (15 rats). Explants were fixed in 10% buffered formalin, routinely processed for histopathology and stained for hematoxylin and eosin, and Giemsa.

Inflammation, vascularization and connective tissue organization were scored semi quantitatively on a scale of 0-4 (none-intense/heavy). At 3 weeks, assessment was made within the implant. At 8 weeks where the implant had disappeared, assessment was made within the remaining granulation tissue at the site.

The thickness of the scar tissue was measured at 100× magnification. Each 10 units of measure equalled 1.28 mm at this magnification.

Two 3-week specimens (both from implants enriched with estrogen) and one 8-week sham specimen were excluded due to errors occurring during histopathological processing.

Data is presented as mean and standard error (SE) and analyzed using the non-parametric Kruskal-Wallis analysis of variance test followed by Mann-Whitney U test for pairwise comparisons between groups.

Results

At 3 weeks, all implants had a satisfactory in-growth of cells. The in-growing cells were distributed throughout the implant. Scores of inflammation differed significantly among different implants. Levels were higher in those enriched with ECM than in plain implants (Table 1). Scores of vascularization, connective tissue organization and thickness of the scar tissue did not differ significantly.

No traces of the implants remained at 8 weeks. There was no foreign body reaction and no signs of a lingering chronic inflammatory reaction. The possible effects of enrichment of the implant had vanished at 8 weeks (Table 2). No significant differences were found in the thickness of the connective tissue after the implants compared to sham sections.

TABLE 1

| n | | Inflammation | Vascularity | Connective tissue | Thickness |
|---|---|---|---|---|---|
| A: Plain implant | 10 | 3.3 (0.15) | 1.9 (0.18) | 0.7 (0.26) | 12.8 (2.3) |
| B: Implant w/ECM | 10 | 3.9 (0.10)* | 1.5 (0.17) | 1.5 (0.27) | 11.8 (1.2) |
| C: Implant w/estrogen | 8 | 3.8 (0.16)** | 1.6 (0.26) | 1.3 (0.37) | 14.9 (2.0) |
| A vs. B vs. C | p = 0.02 | p = 0.33 | p = 0.12 | p = 0.52 | |

3 week scores for inflammation, vascularity and connective tissue organization, 0-4 (none-intense/heavy). Thickness in absolute measure. Mean (standard error).
*A vs. B: p = 0.02,
** A vs. C: p = 0.08

TABLE 2

| n | | Inflammation | Vascularity | Connective tissue | Thickness |
|---|---|---|---|---|---|
| A: Plain implant | 10 | 1.4 (0.16) | 1.5 (0.17) | 3.0 (0.0) | 8.7 (1.3) |
| B: Implant w/ECM | 10 | 1.6 (0.16) | 1.6 (0.16) | 3.0 (0.0) | 9.1 (0.9) |
| C: Implant w/estrogen | 10 | 1.4 (0.16) | 1.6 (0.22) | 3.1 (0.1) | 11.1 (2.2) |
| D: Sham | 9 | 1.0 (0.0) | 0.8 (0.20) | 3.0 (0.0) | 11.6 (2.3) |
| A vs. B vs. C | p = 0.72 | p = 1.0 | p = 1.0 | p = 0.79 | |
| A vs. B vs. C vs. D | | | p = 1.0 | p = 0.87 | |

8 week scores for inflammation, vascularity and connective tissue organization 0-4 (none-intense/heavy). Thickness in absolute measure. Mean (standard error).

Interpretation of Results

The results at 3 weeks indicated a more advanced stage in the healing process in implants enriched with ECM. The initial effects of enrichment with ECM had vanished after 8 weeks.

The MPEG-PLGA implants were completely biocompatible, disappearing in 8 weeks and leaving no trace behind. Qualitatively, the tissue response at 8 weeks was the same after implants as after sham surgery.

The durability of less than 8 weeks was unexpected and is too short for the use per se in pelvic reconstructive surgery. However, due to the characteristics presented here, the implant could have a future role as carrier for cells, such as stem cells or crushed muscle cells, promoting their growth and not affecting the host tissue.

Conclusion

The MPEG-PLGA in all three preparations had excellent biocompatibility. However, the durability was unexpectedly less than 8 weeks, which makes the implant better suitable for use in pelvic reconstructive surgery, if combined with cells, such as stem cells or crushed muscle tissue comprising myoblasts and fibroblasts.

### Example 8

Use of Autologous Muscle Cells and Muscle Fibre Fragments Together with Implant for Pelvic Reconstructive Surgery

A resorbable implant consisting of MPEG-PLGA (methoxypolyethyleneglycol-poly(lactic-co-glycolic acid)) was used. It was freeze-dried and made more hydrophilic to promote in-growth of cells and improve the repairing process. FIG. 16 illustrates the structure.

The aim of the study was to investigate biocompatibility and durability of muscle-derived cells and muscle fibre fragments together with MPEG-PLGA implants to support regeneration of muscle.

Study Design, Materials and Methods

The animal experiments were conducted at the Animal Facility at the Panum Institute, Copenhagen, and approved by the Danish Animal Experiments Inspectorate with permission no. 2009/561-1585.

The experimental animals were 30 Sprague Dawley retired female breeder rats weighing 300-420 grams (Taconic, Denmark). Animal housing and caretaking were provided by Panum Institute according to the national guidelines.

Implants were made of MPEG-PLGA. Three different preparations were used: A) Pure implant, B) Implant with autologous muscle fibre fragments (MFF) C) Implant enriched with autologous muscle progenitor cells (MPC).

Each implant was tested in 10 rats for 3 weeks and in 10 rats for 8 weeks. The rat abdominal subcutaneous model allowed for testing of two pieces of implant per rat.

Rats were anaesthetized with Hypnorm/Dormicum. A 4-cm midline incision was made on the abdomen. After subcutaneous blunt dissection, the implants measuring 10×20×1 mm were placed superficial to the abdominal muscle fascia and tacked in place with one stitch of Vicryl 4-0 (Ethicon). Implants were placed longitudinally to the midline. The skin was closed with Vicryl 4-0. Antibiotic prophylaxis and pain medications were administered according to veterinarian recommendations. Rats were euthanized at 3 and 8 weeks after implantation.

For implants with MFF, a 2-cm incision on the hind leg of the rat was made and a muscle biopsy of 4 mm diameter was taken just before the abdominal surgery. The MFF was prepared in a sterile petri-dish with two scalpels by cutting the biopsy into a fine mash in a drop of physiological saline, while the skin was closed as described above. The implant was placed at the MFF that instantly attached to the implant. At implantation the MFF covered side of the implant faced the fascia.

MPC were grown at Interface Biotech A/S, Denmark, from biopsies obtained as described above, but 2 weeks before surgery.

Isolation and Culture of Muscle-Derived Cells

Two weeks before implantation muscle biopsies were obtained as described above. The biopsies were transferred to sterile transport medium and left overnight at 4° C. Isolation was done according to a modification of “Gene Delivery to Muscle”-protocol {Springer, 2002 250/id}. In brief: the biopsies were minced thoroughly; 0.5 ml collagenase/dispase/CaCl₂ was added and mincing continued; the mixture was incubated at 37° C. for 1 hour; centrifuged for 5 min at 350×g at room temperature; the supernatant was removed; cells were re-suspended in 10 ml F-10 based culture medium and plated in collagen-coated flasks. Cells were seeded in 25 cm² flasks.

After 7 days of culture, the cells were trypsinized and transferred to collagen-coated flasks. After another 7 days of culture, cells were trypsinized, counted and seeded in a concentration of 2×10⁶ cells per implant. Cells were seeded on the implants 24 hours prior to implantation, and the implants with cells were incubated overnight and shipped to the animal facility.

Implants with surrounding full-thickness host tissue were harvested, fixed in 10% buffered formalin and routinely processed for histopathology and immunohistochemistry. The thickness of the sections was 5 μm.

The growth pattern and survival of MPC and MFF was assessed by immunohistochemical staining. In order to identify skeletal muscle cells as opposed to smooth muscle cells, two different primary antibodies were used: monoclonal mouse anti-human desmin (1:100, Clone D33, DAKO, Denmark) and monoclonal mouse anti-human α-Smooth Muscle Actin (SMA) (1:100, Clone 1A4, DAKO). The known cross-reactive specificity of the antibodies with equivalent proteins in rats was confirmed by positive and negative controls. Desmin stains the cytoplasm of skeletal and smooth muscle cells while SMA stains the cytoplasm of smooth muscle cells, but not skeletal muscle cells. As secondary antibody the Histostain®-Plus Kit (InVitrogen) was used. AEC or DAB was used as chromogen for detection of peroxidase activity. Cell nuclei were counterstained with hematoxylin.

If an 8-week-section stained for desmin was negative with regard to MPC and MFF, additional 6 sections, interspaced 30 μm, from that specimen were stained to ensure that there were no remains of MPC or MFF in nearby areas of the specimen.

Results

Surgery was well tolerated in all animals. No erosions or evidence of infection were seen, and there were no signs of implant encapsulation.

Upon explantation, all implants were visible by gross inspection at 3 weeks and none at 8 weeks. In the latter case, a tiny granuloma representing the suture was the only indicator of the implantation site.

The growth pattern was identified by immunohistochemistry with desmin and SMA.

At 3 weeks, the growth pattern of MPC and MFF was qualitatively different upon desmin staining, why quantification of the desmin cells was not carried out. Negative SMA staining of desmin structures in corresponding sections determined that they were of skeletal muscle-type.

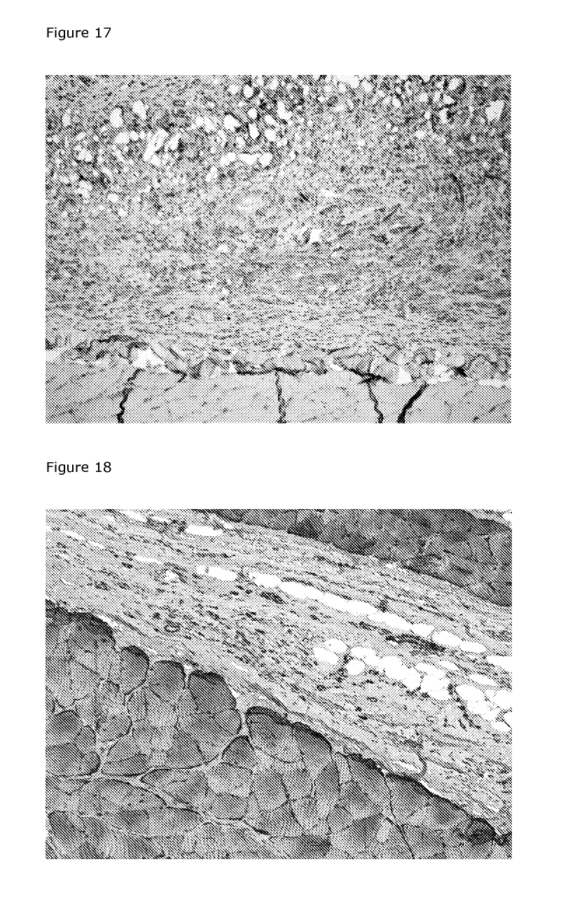

Desmin⁺ cells were observed as being finely distributed within implants seeded with MPC. MFF were identified as fragmented muscle tissue with striation unevenly distributed beneath the implants (FIG. 17). The MPEG-PLGA had unspecific staining for desmin in varying degrees, why the morphology was the key factor in the interpretation of the staining.

In one of the pure implants, desmin⁺ cells were found in a pattern similar to that of implants seeded with MPC, however to a lesser extent.

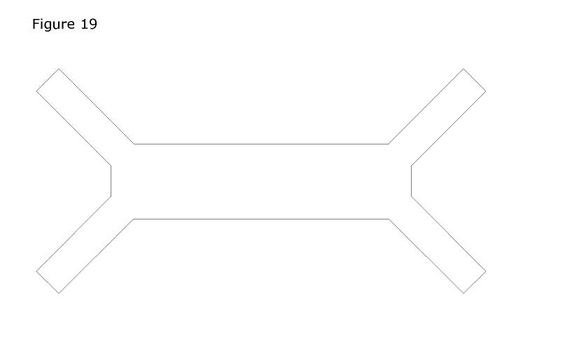

At 8 weeks, the MFF had survived and were found as fragmented striated muscle tissue in 6 of the 10 specimens (FIG. 18). Further two were doubtful because the morphology and position of the desmin⁺ structures were different: It could be artifacts representing distorted/twisted skin muscle. In one specimen, a homogenous faintly positive area was found, probably representing dead MFF eaten by macrophages. One specimen was completely negative.

The MPEG-PLGA itself and the MPC had vanished at 8 weeks.

In conclusion, the MPEG-PLGA in all three preparations had excellent biocompatibility and disappeared within 8 weeks in this abdominal rat model. When autologous muscle progenitor cells were combined with MPEG-PLGA skeletal muscle cells were identified after 3 weeks but not after 8 weeks. In contradiction, skeletal muscle was identified both after 3 and 8 weeks when MPEG-PLGA was combined with fragmented muscle fibres. This shows a high survival rate for the muscle fibres when combined with MPEG-PLGA.

1-58. (canceled)

59. A biodegradable surgical implant for support, augmentation and regeneration of living tissue in a subject, comprising
a) a synthetic biodegradable homogenous sheet of scaffold,
b) one or more biodegradable reinforcing members.

60. The biodegradable surgical implant according to claim 59, wherein said synthetic biodegradable homogenous sheet of scaffold is hydrophilic.

61. The biodegradable surgical implant according to claim 59, wherein said synthetic biodegradable homogenous sheet of scaffold has the ability to, within 5 minutes, such as within 2 minutes at 30° C., absorb water in an amount of at least 10%, such as at least 20%, such as at least 30%, such as at least 50% of the scaffold volume.

62. The biodegradable surgical implant according to claim 59, wherein the volume % of said reinforcing member is less than 40% of the implant.

63. The biodegradable surgical implant according to claim 59, wherein said synthetic biodegradable homogenous sheet of scaffold is prepared by freeze-drying.

64. The biodegradable surgical implant according to claim 59, wherein said biodegradable reinforcing member is based on fibres and/or threads with a thickness of about 10 nm-1000 μm, such as in the range of about 10 nm-800 μm, such as in the range of about 10 nm-500 μm.

65. The biodegradable surgical implant according to claim 59, wherein said biodegradable reinforcing member is a sheet made of a woven fabric, knitted fabric, mesh, non-woven felt, made of filaments or fibres.

66. The biodegradable surgical implant according to claim 59, wherein said synthetic biodegradable homogenous sheet of scaffold is completely degradable within 1-48 months, such as 4-36, such as 6-24, or 1-12 months of in situ application.

67. The biodegradable surgical implant according to claim 59, wherein said biodegradable reinforcing member promotes cell attachment and in-growth of cells derived from the living tissue in said subject or from the application of cell or tissue explants.

68. The biodegradable surgical implant according to claim 59, wherein said reinforced biodegradable member is made from a polymer of poly(lactide-co-glycolide) PLGA, such as a polymer wherein the molar ratio of (i) lactide units and (ii) glycolide units in the poly(lactide-co-glycolide) residue is in the range of 90:10 to 10:90, such as in the range of 80:20 to 10:90, such as about 10:90.

69. The biodegradable surgical implant according to claim 59, wherein said synthetic biodegradable homogenous sheet of scaffold is a polymer of the general formula:
A-O—(CHR¹CHR²O)ₙ—B
wherein;
A is a poly(lactide-co-glycolide) residue of a molecular weight of at least 4000 g/mol, the molar ratio of (i) lactide units and (ii) glycolide units in the poly(lactide-co-glycolide) residue being in the range of 80:20 to 10:90;
B is either a poly(lactide-co-glycolide) residue as defined for A or is selected from the group consisting of hydrogen, C₁₋₆-alkyl and hydroxy protecting groups,
one of R¹ and R² within each —(CHR¹CHR²O)— unit is selected from hydrogen and methyl, and the other of R¹ and R² within the same —(CHR¹CHR²O)— unit is hydrogen;
n represents the average number of —(CHR¹CHR²O)— units within a polymer chain and is an integer in the range of 10-1000; and
wherein
the molar ratio of (iii) polyalkylene glycol units —(CHR¹CHR²O)— to the combined amount of (i) lactide units and (ii) glycolide units in the poly(lactide-co-glycolide) residue(s) is at the most 20:80;
and wherein the molecular weight of the copolymer is at least 10,000 g/mol, preferably at least 15,000 g/mol.

70. The biodegradable surgical implant according to claim 69, wherein the weight percentage of (iii) polyalkylene glycol units —(CHR¹CHR²O)— to the combined amount of (i) lactide units and (ii) glycolide units in the poly(lactide-co-glycolide) residue(s) is in the range of 4%-10% w/w.

71. The biodegradable surgical implant according to claim 59, wherein said synthetic biodegradable homogenous sheet of scaffold is prepared by freeze-drying a solution comprising the biodegradable polymer in solution.

72. The biodegradable surgical implant according to claim 59, which implant further comprises, within said scaffold, one or more components which facilitate the cell adhesion and/or in-growth for regeneration of tissue, such as a component selected from the group consisting of: estrogen, estrogen derivatives, thrombin, ECM powder, chondroitin sulfate, hyaluronan, hyaluronic acid (HA), heparin sulfate, heparan sulfate, dermatan sulfate, growth factors, fibrin, fibronectin, elastin, collagen, such as collagen type I and/or type II, gelatin, and aggrecan, or any other suitable extracellular matrix component.

73. The biodegradable surgical implant according to claim 59, which implant further comprises, within said scaffold, one or more components selected from the group consisting of growth factors, such as Insulin-like growth factors (IGFs), such as IGF-1 or IGF-2, or Transforming growth factors (TGFs), such as TGF-alpha or TGF-beta, or Fibroblast growth factors (FGFs), such as FGF-1 or FGF-2, or Platelet-derived growth factors (PDGFs), such as PDGF-AA, PDGF-BB or PDGF-AB, or Nerve growth factor (NGF), or Human growth hormone (hGH), and Mechano Growth Factor (MGF).

74. The biodegradable surgical implant according to claim 59, which implant further comprises, within said scaffold, a sample of cells or tissue explants.

75. T The biodegradable surgical implant according to claim 59, which implant is formed as a tube and/or comprises a flap and/or a pocket suitable for application of a suspension of a sample of cells or tissue explants to said implant.

76. The biodegradable surgical implant according to claim 59, which implant comprises two or more separated pieces of synthetic biodegradable homogenous sheets of scaffold, such as 3, 4, 5 or 6 pieces of synthetic biodegradable homogenous sheets of scaffold attached to a reinforcing member, such as a mesh of a different polymer.

77. The biodegradable surgical implant according to claim 59, which implant comprises two or more, such as 4 or 6 arms or extensions for attachment to structures in the site of implantation, such as in the pelvic region.

78. A method for support, augmentation and regeneration of living tissue within a subject, said method comprising implantation of a biodegradable surgical implant comprising a synthetic biodegradable scaffold together with a sample of autologous cells or tissue explants within said subject at the site wherein support, augmentation and regeneration of living tissue is required.

79. The method according to claim 59, wherein said synthetic biodegradable scaffold is a homogenous sheet.

80. A method according to claim 59, wherein said biodegradable surgical implant is according to claim 59.

81. The method according to claim 78, wherein said subject is suffering from a medical prolapse, such as pelvic organ prolapse, or hernia, or stress urinary incontinence.

82. A method for the preparation of a biodegradable surgical implant comprising a synthetic biodegradable scaffold and autologous cells or tissue explants of a subject, suitable for support augmentation and regeneration of living tissue within said subject, said method comprising ex vivo application of a sample of said autologous cells or tissue explants on or within said biodegradable surgical implant comprising a synthetic biodegradable scaffold prior to implantation within said subject at the site wherein support, augmentation and regeneration of living tissue is required.

83. The method according to claim 78, wherein the amount of cells in said sample of cells or tissue explants used is in the range of about 0.1×10⁴ cells to about 10×10⁶ cells per cm² of implant.

84. The method according to claim 78, wherein the tissue explants is from muscle tissue, stem cells, such as stem cells capable of differentiation into myoblasts, or fibroblasts; or combinations thereof.

85. The method according to claim 78, wherein said cells or tissue explants are derived from a human.

86. The method according to claim 78, wherein said cells or tissue explants are not cultured in vitro prior to implantation.

87. The method according to claim 78, wherein said cells or tissue explants are harvested and used according to the method in the operating room.

88. The method according to claim 78, which method further comprises application to said biodegradable surgical implant of a composition comprising a component which facilitates the cell adhesion and/or in-growth for regeneration of tissue, such as a component selected from the group consisting of: estrogen, estrogen derivatives, thrombin, ECM powder, chondroitin sulfate, hyaluronan, hyaluronic acid (HA), heparin sulfate, heparan sulfate, dermatan sulfate, growth factors, fibrin, fibronectin, elastin, collagen, such as collagen type I and/or type II, gelatin, and aggrecan, or any other suitable extracellular matrix component.

89. The method according to claim 78, which method further comprises application to said biodegradable surgical implant of a composition comprising a component selected from the group consisting of growth factors, such as Insulin-like growth factors (IGFs), such as IGF-1 or IGF-2, or Transforming growth factors (TGFs), such as TGF-alpha or TGF-beta, or Fibroblast growth factors (FGFs), such as FGF-1 or FGF-2, or Platelet-derived growth factors (PDGFs), such as PDGF-AA, PDGF-BB or PDGF-AB, or Nerve growth factor (NGF), or Human growth hormone (hGH), and Mechano Growth Factor (MGF).

90. A biodegradable surgical implant comprising a synthetic biodegradable scaffold for use in a method for support, augmentation and regeneration of living tissue within a subject, said method comprising implantation of said biodegradable surgical implant comprising a synthetic biodegradable scaffold together with a sample of autologous cells or tissue explants within said subject at the site wherein support, augmentation and regeneration of living tissue is required.

91. A biodegradable surgical implant comprising a synthetic biodegradable scaffold; for use in a method for support, augmentation and regeneration of living tissue within a subject, said method comprising the steps of (i) extracting a tissue sample from the subject; (ii) disintegration or disruption of the tissue sample; (iii) implanting the scaffold and the crushed tissue sample into the subject.

92. The biodegradable surgical implant according to claim 91, wherein said disintegration or disruption is done by crushing the tissue sample in a device comprising holes or a mesh for crushing a tissue sample by the application of pressure by which the tissue sample is forced through said mesh or holes.

93. A kit comprising
a) a biodegradable surgical implant comprising a synthetic biodegradable scaffold;
b) a sample of autologous cells or tissue explants; and
c) optionally instructions for use in a method for support, augmentation and regeneration of living tissue within a subject, such as in a subject with a medical prolapse, such as rectal or pelvic organ prolapse, or hernia, said method comprising implantation of said biodegradable surgical implant together with an autologous sample of cells or tissue explants within said subject at the site wherein support, augmentation and/or regeneration of living tissue is required.

94. A kit comprising
a) a synthetic biodegradable scaffold; and
b) a device suitable for disintegration or disruption of a tissue sample.

95. The kit according to claim 94, wherein said device suitable for disintegration or disruption comprises holes or a mesh for crushing said tissue sample by the application of pressure by which the tissue sample is forced through said mesh or holes.

96. The kit according to claim 94, wherein said device suitable for disintegration or disruption is based on a mill, ultra sonic treatment, homogenizer, high pressure, or physical force from knives or other instruments.

97. A kit comprising:
(a) a biodegradable surgical implant comprising the synthetic biodegradable scaffold of claim 59;
(b) a sample of autologous cells or tissue explants; and optionally
(c) instructions for use in a method for support, augmentation and regeneration of living tissue within a subject.
